(19) United States
(12) Patent Application Publication
(10) Pub. No.: US 2018/0153420 A1
(43) Pub. Date: Jun. 7, 2018

(54) APPARATUS AND METHOD FOR OPTICAL MEASUREMENT OF CARDIOVASCULAR FITNESS, STRESS AND PHYSIOLOGICAL PARAMETERS

(71) Applicant: ELFI-TECH LTD., Rehovot (IL)

(72) Inventors: Ilya FINE, Rehovot (IL); Alexander Kaminsky, IL (IL)

(21) Appl. No.: 15/811,823

(22) Filed: Nov. 14, 2017

Related U.S. Application Data
(63) Continuation-in-part of application No. 14/503,395, filed on Sep. 30, 2014, now abandoned.
Application No. 14/503,395 is a continuation of application No. PCT/IB2015/001157, filed on May 21, 2015.
(60) Provisional application No. 61/884,975, filed on Sep. 30, 2013.
(60) Provisional application No. 61/884,202, filed on Sep. 30, 2013.

(51) Int. Cl. A61B 5/024; A61B 5/02; A61B 5/026; A61B 5/00; A61B 5/022

(52) U.S. Cl. CPC A61B 5/02416; A61B 5/02035; A61B 5/1455; A61B 5/7239; A61B 5/02233; A61B 5/0261

(57) ABSTRACT

Apparatus and methods for optical and non-invasive measurement of cardiovascular fitness and/or stress and/or physiological parameters are disclosed herein.

### CROSS-REFERENCE TO RELATED APPLICATIONS

The present application is a continuation of PCT/IB2015/001157 filed on May 21, 2015 which is incorporated by reference in its entirety. The present application is also a continuation-in-part of U.S. patent application Ser. No. 14/503,395 filed Sep. 30, 2014, which is incorporated by reference in its entirety. U.S. patent application Ser. No. 14/503,395 claims priority to U.S. 61/884,975 patent application number filed Sep. 30, 2013 and to U.S. 61/884,202 filed Sep. 30, 2013, both of which are incorporated by reference in their entirety.

### BACKGROUND

### Cardiovascular Fitness Parameters

Fitness and stress management are important components for a healthy lifestyle. Periodic and frequent measurements of fitness and stress level are recommended to be applied as an effective way to recognize early signs of risk and for preventing many chronic diseases and condition. Recently a large percentage of population started to uses smartphones and wearable devices. The very existence of this new platform enables to introduce new sensor solutions, where the processing capabilities of these devices can be used for new applications. Currently some of smartphones and wearable watches include the PPG sensors capable of measuring the pulse rate and R wave-to-R wave (R-R) intervals from the finger or wrist.

Cardiovascular fitness represents the efficiency of the heart, lungs and vascular system in delivering oxygen to the working muscles so that prolonged physical work can be maintained. Many fitness and wellness programs aim to improve the cardiovascular strength and endurance. In order to measure cardiovascular strength of a subject the different stress tests are used. For example, the Bruce Protocol Treadmill test is used for evaluating cardiac fitness. As the Bruce Protocol Treadmill test is a maximal fitness test, one has to run continuously until get tired. The goal is to increase workload incrementally to induce ischemia or until a predetermined workload is reached. The main disadvantage of these test is, that it requires using a treadmill and ECG equipment. The test is time and energy consuming and not very suitable for home for performing at daily basis. In addition, this test is based on subjective feeling of triteness and the related physiological parameters are not measured directly. Nowadays, this type of test can be performed and analyzed under professional supervision, during the exercise tolerance stress testing

Obviously, such a test cannot be used as a substitute for daily management of fitness or stress level of subjects.

Thanks to Photoplethysmography (PPG) sensors, with assistance of the smartphones and wearable devices the pulse rate measurement can be performed simlessly at home. The improvement of the endurance or to make kind of a pre-diagnostic assessment is to measure the heart rate (HR) post stress recovery pattern. Post-exercise heart rate recovery, though a readily obtainable parameter and a powerful and independent predictor of cardiovascular and all-cause mortality in healthy adults and in those with cardiovascular diseases, is often overlooked as an indicator of cardiovascular fitness. Heart rate recovery (HRR) is thought to be major characteristics of parasympathetic reactivation but not providing comprehensive information regarding the endurance improvement

Unfortunately, the HR and HRR pattern by themselves do not provide comprehensive information about the cardiovascular ability to supply the blood but rather indirect indications via the regulatory mechanics. Maximal heart rate is not an appropriate indicator of the amount of blood being pumped around the body, especially not for trained endurance athletes.

All tasks related to the heart rate are not addressing the heart pump systolic and diastolic performance during the cycle of heart beat. The heart cycle starts from the systolic cession. Systole refers to the contraction of the left ventricle, which drives blood into the aorta. Diastole covers a period when the left and right ventricles are relaxed. Systolic and diastolic dysfunction can cause congestive heart failure (CHF). Stroke volume is defined as the volume of blood pumped by the heart with each beat. Unlike heart rate, SV provides a direct indication of the heart performance. If SV increases with endurance training, it means that more blood is pumped around the body with every heart bit beat. Therefore, a reduction in maximal heart rate does not result in the body receiving less blood. In fact, the opposite is true, as the reduced heart rate is more than compensated for by higher stroke volume. The reason for increased stroke volume is an increase in the end diastolic volume), the volume of blood in the left ventricle just before contraction. The major difference in the endurance-trained heart is a bigger stroke volume. The trained heart gets bigger and pumps more blood each.

### Blood Pressure Wave-Forms

Invasive blood pressure (IBP) is a method of measuring blood pressure internally by using a sensitive IV catheter inserted into an artery. This provides a more accurate reading of the patent's current blood pressure.

The blood-pressure-waveform generated by IBP is the ‘gold standard’ of blood pressure waveforms. As shown in FIG. 1A, this waveform includes various features including the ‘peak,’ the reflected-wave marker (i.e. indicated by an inflection point and/or a protrusion), the dicrotic notch and the vascular resistance wave marker (i.e. indicated by an inflection point and/or protrusion).

The IBP waveform is considered the ‘gold standard’ blood pressure waveform. Although the waveform from non-invasive techniques such as PPG resemble the IBP waveform in general terms, comparing of FIG. 1B to FIG. 1A illustrates that a great deal of information is lost.

### Dynamic Light Scattering (DLS) for Non-Invasive In-Vivo Measurement of Biological Parameters

WO 2008/053474 and WO2012064326, each of which are incorporated herein by reference in its entirety, each disclose a system and method for in vivo measurement of biological parameters by dynamic light scattering techniques.

In particular, WO 2008/053474 discloses a novel optical technique suitable for the in vivo measurement in a subject utilizing dynamic light scattering (DLS) approach. The effect of DLS are utilized for the measurement of variety of blood related parameters, such as viscosity of the blood and blood plasma, blood flow, arterial blood pressure and other blood chemistry and rheology related parameters such as concentration of analyte (e.g. glucose, hemoglobin, etc.), oxygen saturation etc.

DLS is a well-established technique to provide data on the size and shape of particles from temporal speckle analysis. When a coherent light beam (laser beam, for example) is incident on a scattering (rough) surface, a time-dependent fluctuation in the scattering property of the surface and thus in the scattering intensity (transmission and/or reflection) from the surface is observed. These fluctuations are due to the fact that the particles are undergoing Brownian or regular flow motion and so the distance between the particles is constantly changing with time. This scattered light then undergoes either constructive or destructive interference by the surrounding particles and within this intensity fluctuation information is contained about the time scale of movement of the particles. The scattered light is in the form of speckles pattern, being detected in the far diffraction zone. The laser speckle is an interference pattern produced by the light reflected or scattered from different parts of an illuminated surface. When an area is illuminated by laser light and is imaged onto a camera, a granular or speckle pattern is produced. If the scattered particles are moving, a time-varying speckle pattern is generated at each pixel in the image. The intensity variations of this pattern contain information about the scattered particles. The detected signal is amplified and digitized for further analysis by using the autocorrelation function (ACF) technique. The technique is applicable either by heterodyne or by a homodyne DLS setup.

The kinetics of optical manifestations of two kinds of physiological signals is measured in vivo: the pulsatile signal associated with heart beats and the post-occlusion optical signal which is induced by an artificially generated blood flow cessation. The light transmission and/or reflection signals are used as a control of the physiological response. This kind of control measurement can be carried out simultaneously with the DLS reflection measurement. The mutual correspondence between DLS and standard optical signals is subject to a comparison analysis.

Reference is now made to FIGS. 2A-2B. FIG. 2A, taken from WO 2008/053474 (and slightly modified) illustrates an apparatus for performing a DLS measurement. A coherent light source (e.g. a vertical-cavity surface-emitting laser (VCSEL)) emits coherent light to illuminate the skin (step S201)—this coherent light scatters off of red blood cells (RBCs) within blood vessels of the skin (or beneath the skin) to induce a scattered-light optical response The optical response is detected (step S205) by photodetectors to generate an electrical signal descriptive of the scattered-light optical response. This electrical signal is processed (e.g. using autocorrelation or power spectrum analysis) (step S213) to produce a time-dependent blood-shear-rate descriptive signal. One or physiological parameters (e.g. pulse rate or blood pressure) are computed from the blood-shear-rate-descriptive signal.

Red blood cells (RBSs) suspended within blood plasma do not travel at the same velocity—the blood-shear-rate-descriptive signal describes differences in velocities of red-blood-cells suspended in the blood plasma. In certain frequency domains, blood-shear is primarily due to pulse. By illuminating skin, collecting scattered light and subjecting the scattered light to speckle analysis (e.g. to analyze temporal fluctuations of speckle patterns), it is possible to derive a signal descriptive of a blood-shear over a cross section of blood vessel(s) and/or over a ensemble of blood vessels.

FIG. 2C, taken from WO 2008/053474, illustrates one example of a scattered-light time-dependent optical response signal. FIG. 2D illustrates one example of a time-dependent blood-shear-rate descriptive signal.

Although the signal is adequate for detecting various physiological parameters disclosed in WO 2008/053474, the signal of FIG. 2D also surfers from a ‘loss of information (i.e. similar to the PPG signal of FIG. 1B) about the blood pressure waveform relative to the IBP signal of FIG. 1A.

### SUMMARY

A method for optically measuring a cardiovascular fitness and/or stress and/or physiological parameter specific to a mammalian subject, the method comprising: a. illuminating a portion of the subject's skin to scatter partially or entirely coherent light off of moving red blood cells (RBCs) of the subject to induce a scattered-light time-dependent optical response; b. receiving the scattered light by a photodetector(s) to generate an electrical signal descriptive of the induced scattered-light time-dependent optical response; c. processing the scattered-light-optical-response-descriptive electrical signal or a product thereof to generate therefrom a time-dependent blood-shear-rate-descriptive signal wherein the processing is performed according to a function-transformation-algorithm that is dynamically adjusted over time in response to (i) a measured or predicted similarity between the time-dependent blood-shear-rate-descriptive signal and a blood-pressure-waveform; (ii) a measured or predicted presence or strength of blood-pressure-waveform-feature(s) within the time-dependent blood-shear-rate-descriptive signal; and d. computing the cardiovascular fitness and/or stress and/or physiological parameter from the time-dependent blood-shear-rate-descriptive signal.

A method for optically measuring a cardiovascular fitness and/or stress and/or physiological parameter of a subject, the method comprising: a. illuminating a portion of the subject's skin to scatter partially or entirely coherent light off of moving red blood cells (RBCs) of the subject to induce a scattered-light time-dependent optical response; b. receiving the scattered light by a photodetector(s) to generate an electrical signal descriptive of the induced scattered-light time-dependent optical response; c. for each transformation-function-algorithm of a plurality of transformation-function-algorithms, respectively processing the scattered-light-optical-response-descriptive electrical signal or a product thereof to generate therefrom a respective time-dependent blood-shear-rate-descriptive signal; d. analyzing each time-dependent blood-shear-rate-descriptive signal to determine a presence or strength of pulsatile-waveform-feature(s) within the time-dependent blood-shear-rate-descriptive signal; e. comparing the results of the analysis of each time-dependent blood-shear-rate-descriptive signal; and f. computing, in accordance with the results of the comparing and from one or more of the time-dependent blood-shear-rate-descriptive signal or a mathematical function thereof, the cardiovascular fitness and/or stress and/or physiological parameter of the subject.

In some embodiments, the pulsatile-waveform-feature(s) any of the following parameters, or a relation therebetween: (i) a presence, temporal-location, shape or amplitude of a reflected-wave protrusion; (ii) a presence, temporal-location, shape or amplitude of a dicrotic notch; (iii) a presence, temporal-location, shape or amplitude of a vascular resistance wave protrusion; (iv) a systolic down-slope; (v) a diastolic down-slope; (v) a systolic upstroke slope; (vi) relative heights or time-delays between of any of an overall peak, reflected-wave marker, a dicrotic notch, and vascular resistance wave marker.

In some embodiments, the subject-specific parameter is selected from the group consisting of (i) a pulse; (ii) a heart-rate variability; (iii) a blood pressure; (iv) a stroke-volume; (iv) respiration rate; (iv) an apnea event; (v) a measure of the aortic valve functionality; (vi) the sympathetic system activity, systolic blood pressure, and vascular aging; (vii) a measure of the myocardium's ability to expel blood to the body height of the Dicrotic notch relative to the systolic peak, and (viii) the time delay between them; a measure of arterial stiffness and ability to resist blood flow; (ix) a measure of how fast myocardium relaxes at the end of systolic cycle; (x) a measure of how fast the myocardium relaxes at the end of diastole; (xi) a measure of stroke volume.

In some embodiments, i. the scattered light is received by first and second photodetectors respectively situated at first and second locations to respectively generate first and second scattered-light-optical-response-descriptive electrical signals; and ii. the time-dependent blood-shear-rate-descriptive signal is derived from a difference between the first and second scattered-light-optical-response-descriptive electrical signals.

In some embodiments, the processing comprises subjecting the scattered-light-optical-response-descriptive electrical signal or a product thereof to at least one of an autocorrelation analysis and a power spectrum analysis.

A method for optically measuring a cardiovascular fitness and/or stress and/or physiological parameter of a subject, the method comprising: a. illuminating a portion of the subject's skin to scatter partially or entirely coherent light off of moving red blood cells (RBCs) of the subject to induce a scattered-light time-dependent optical response; b. receiving the scattered light by a photodetector(s) to generate an electrical signal descriptive of the induced scattered-light time-dependent optical response; c. processing the scattered-light-optical-response-descriptive electrical signal or a product thereof to generate therefrom a time-dependent blood-shear-rate-descriptive signal; and d. post-processing the time-dependent blood-shear-rate-descriptive signal by computing therefrom at least one or more of: (i) a systolic upstroke slope; (ii) a systolic downstroke slope; (iii) a diastolic downstroke slope; (iv) a stroke-volume; (v) a time-interval between a pulse-peak and a peak of the reflected wave; (vi) a time-interval between a pulse-peak and a time of the dicrotic notch; (v) a time-interval between a pulse-peak and a time of the vascular-resistance wave; (v) a time-interval between a peak of the reflected wave and a time of the dicrotic notch; (vi) a time-interval between a marker of the reflected wave and a time of the vascular-resistance wave; and (vii) a time-interval between a time of the dicrotic notch and a time of the vascular-resistance wave.

A method for optically measuring a fitness and/or stress and/or physiological parameter of a subject, the method comprising: a. illuminating a portion of the subject's skin to scatter partially or entirely coherent light off of moving red blood cells (RBCs) of the subject to induce a scattered-light time-dependent optical response; b. receiving the scattered light by a photodetector(s) to generate an electrical signal descriptive of the induced scattered-light time-dependent optical response; c. processing the scattered-light-optical-response-descriptive electrical signal or a product thereof to generate therefrom a time-dependent blood-shear-rate-descriptive signal; d. for each given cardiac cycle of a plurality of cardiac cycles, obtaining or computing respective cardiac-cycle signal-form characteristics of the patient; e. generating a multi-cycle blood-shear-rate-signal-derived cardiac-parameter stroke volume data set by computing, for each given cardiac cycle of the plurality of cardiac cycles, a respective cardiac-cycle-specific stroke-volume parameter by subjecting the time-dependent blood-shear-rate-descriptive signal to a temporal analysis in accordance with the respective cardiac-cycle signal-form characteristics specific for the given cardiac cycle; f. generating a multi-cycle pulse-rate data set by measuring, for each given cardiac cycle of the plurality of cardiac cycles, a respective pulse-rate specific for the given cardiac cycle; and g. quantifying a correlation between the multi-cycle stroke-volume parameter data set and the multi-cycle pulse rate data set; and h. computing the cardiovascular recovery metric of the subject from the quantified magnitude.

A method for optically measuring a fitness and/or stress and/or physiological parameter of a subject, the method comprising: a. illuminating a portion of the subject's skin to scatter partially or entirely coherent light off of moving red blood cells (RBCs) of the subject to induce a scattered-light time-dependent optical response; b. receiving the scattered light by a photodetector(s) to generate an electrical signal descriptive of the induced scattered-light time-dependent optical response; c. processing the scattered-light-optical-response-descriptive electrical signal or a product thereof to generate therefrom a time-dependent blood-shear-rate-descriptive signal; d. for each given cardiac cycle of a plurality of cardiac cycles, obtaining or computing respective cardiac-cycle signal-form characteristics of the patient; e. generating a multi-cycle blood-shear-rate-signal-derived cardiac-parameter stroke volume data set by computing, for each given cardiac cycle of the plurality of cardiac cycles, a respective cardiac-cycle-specific stroke-volume parameter by subjecting the time-dependent blood-shear-rate-descriptive signal to a temporal analysis in accordance with the respective cardiac-cycle signal-form characteristics specific for the given cardiac cycle; f. temporally analyzing the stroke-volume-parameter signal to characterize temporal fluctuations thereof; and g. computing the respiratory rate from the characterized temporal fluctuations.

Embodiments of the invention relate to methods and apparatus of optically and non-invasively (i) measuring a cardiovascular recovery metric or a respiration rate and (ii) detecting certain events related to respiration rate or a change therein. In particular, it is possible to measure a blood-shear parameter and to derive the cardiovascular recovery metric or reparation rate therefrom.

Some embodiments relate to a cardiovascular recovery metric. When a subject exercise or subjects his/her cardiovascular system to an elevated load, his/her pulse rate increases and his/her stroke volume parameter increases. After the exercise ceases, or decreases in intensity, his/her cardiovascular system no longer has the same need to oxygenate the body as was previously required during more intense activity. As a result, the cardiovascular system returns to a lower rate of activity. However, the amount of time required for this to occur differs between subjects. More fit subjects tend to have a significantly lower recovery time than those subjects who are not in shape (e.g. overweight, unaccustomed to exercise, etc).

Knowledge of the cardiovascular recovery time may be useful in diagnosing or prognosticating heart disease, and there is a need to encourage people to measure this physiological parameter. Unfortunately, it may require a certain amount of time to accurately measure this ‘recovery time’ (for example, minutes) and many subjects are unlikely to comply—instead, this parameter may just go unmeasured.

Embodiments of the invention relate to an apparatus and method for accurately, quickly, optically and non-invasively measuring the cardiovascular recovery time.

In particular, it is now disclosed that the correlation between a per-cycle stroke-volume and the per-cycle pulse rate may be measured (e.g. using any apparatus disclosed herein), and the cardiovascular recovery metric may be computed therefrom. This topic will be further discussed below.

Some embodiments relate to respiration rate. In particular, it is now disclosed an apparatus and method which (i) monitor cardiac-cycle specific stroke volume parameters to compute a stroke volume parameter signal; and (ii) subject this stroke volume parameter signal to a temporal analysis. The respiration rate may be derived from the temporal analysis—e.g. by computing a dominant frequency.

### BRIEF DESCRIPTION OF THE DRAWINGS

FIGS. 1A-1B respectively illustrate prior art examples of IBP and PPH signals.

FIGS. 2A-2B respectively illustrate an apparatus and a method for performing a DLS measurement.

FIG. 2C illustrates one example of a scattered-light time-dependent optical response signal.

FIG. 2D illustrates one example of a time-dependent blood-shear-rate descriptive signal.

FIG. 3A schematically illustrates a time-dependent blood-shear-rate descriptive DLS signal.

FIG. 3B illustrates actual signals that were computed according to presently-disclosed techniques.

FIGS. 4A-4D illustrate examples of transforming a scattered-light time-dependent optical response signal into a time-dependent blood-shear-rate descriptive signal.

FIG. 5 is a flow-chart of a technique for computing a time-dependent blood-shear-rate descriptive signal and/or a physiological parameter derived therefrom.

FIG. 6 illustrates another variation of blood-pressure-waveform feature(s) for scoring time-dependent blood-shear-rate descriptive signal.

FIG. 7A illustrates overlapping time-windows.

FIG. 7B illustrates non-overlapping time-windows.

FIG. 8 is a flow chart illustrating a method according to some embodiments of the invention.

FIG. 9 is an illustration of a DLS measurement based system for measuring one or more blood parameters.

FIG. 10 is a flow chart of a technique for computing a cardiac-cycle specific stroke volume parameter of a subject.

FIG. 11 illustrates signal form characteristics.

FIGS. 12A-12B relate to correlating between the per-cycle stroke volume parameter and the per-cardiac-cycle pulse rate.

FIGS. 13A-13B illustrate the quantifying a correlation between cycle-specific heart rate (y axis) and cycle-specific stroke volume parameter.

FIGS. 14A-14B relate to a second application of the technique for deriving the per-cycle stroke volume parameter.

FIG. 14C is a flow chart illustrating a method according to some embodiments of the invention.

FIG. 15 relates to the technique of FIG. 14A.

FIG. 16 illustrates a velocity profile of flowing blood in small vessels.

FIG. 17 demonstrates how the ACF looks like for the pulsatile signal measured by using mDLS.

FIG. 18 demonstrates the typical change of autocorrelation function (ACF) of the DLS signal being measured from the finger tip.

FIG. 19 illustrates low frequency fluctuations associated with a blood pressure modulations which are associated with the sympathetic nervous system activity.

FIG. 20 is an example of identification of onset, peak and dischrotic notch of the pulse-wave.

### DESCRIPTION OF EMBODIMENTS

The claims below will be better understood by referring to the present detailed description of example embodiments with reference to the figures. The description, embodiments and figures are not to be taken as limiting the scope of the claims. It should be understood that not every feature of the presently disclosed methods, apparatuses, and computer readable media having stored thereon computer code for logical protocol command disambiguation is necessary in every implementation. It should also be understood that throughout this disclosure, where a process or method is shown or described, the steps of the method may be performed in any order or simultaneously, unless it is clear from the context that one step depends on another being performed first. As used throughout this application, the word “may” is used in a permissive sense (i.e., meaning “having the potential to’), rather than the mandatory sense (i.e. meaning “must”).

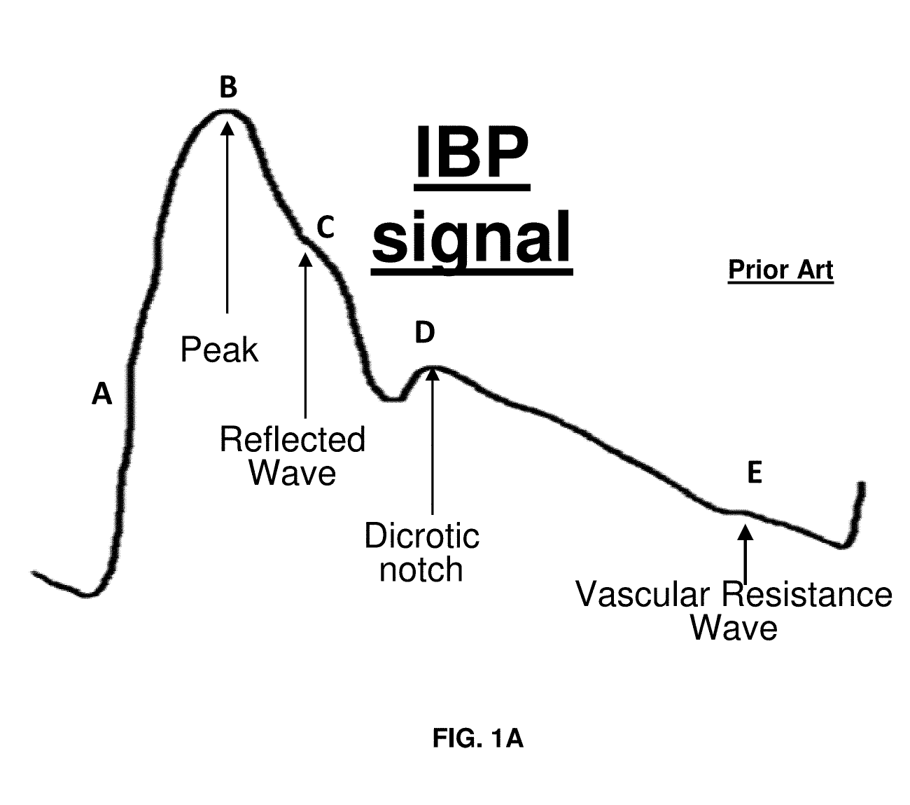

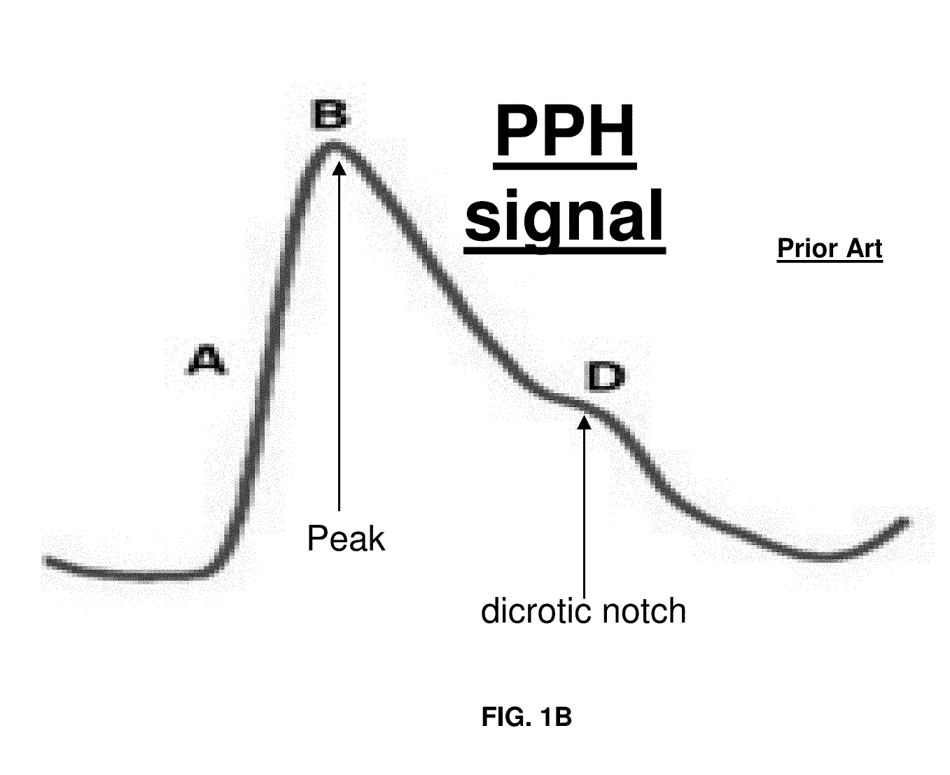

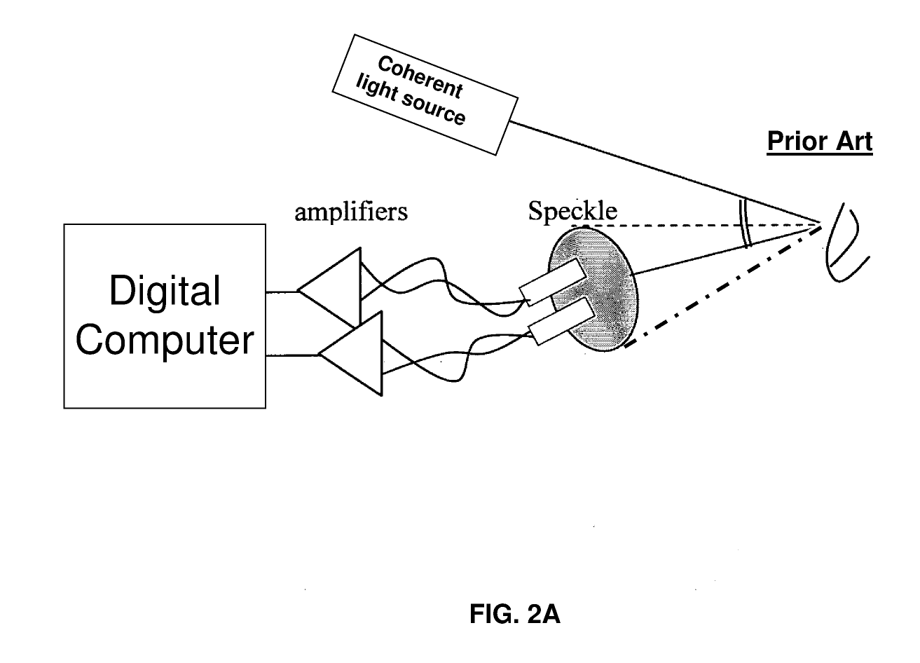

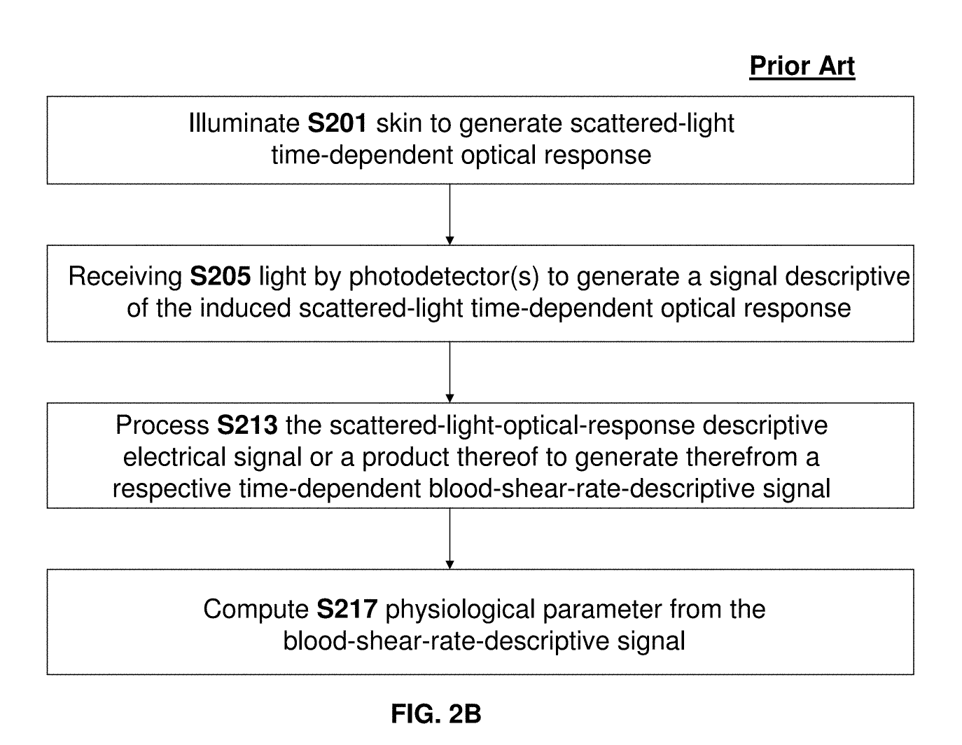

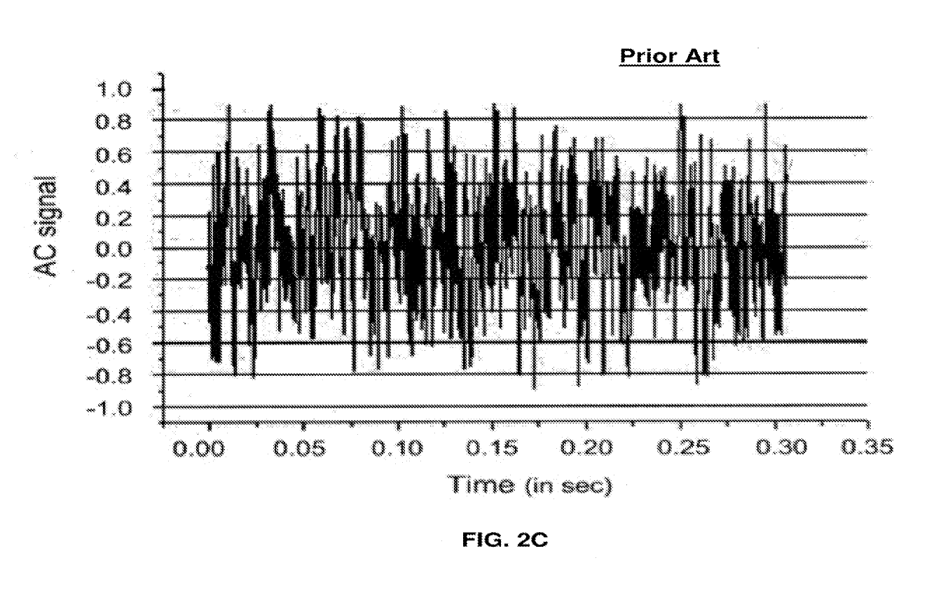

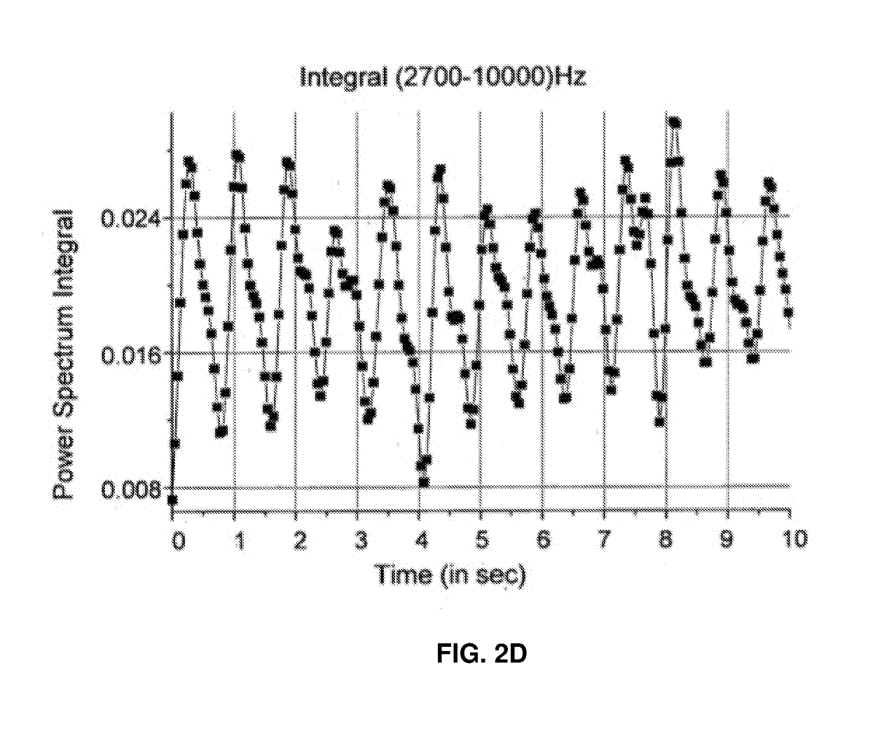

Embodiments of the present invention relate to improved techniques for processing the scattered-light time-dependent optical response signal to yield a time-dependent blood-shear-rate descriptive signal that more closely resembles the ‘gold-standard’ invasive blood pressure waveform of FIG. 1A, and does not suffer from the ‘loss of information’ evident upon inspection of FIG. 1B and FIG. 2D.

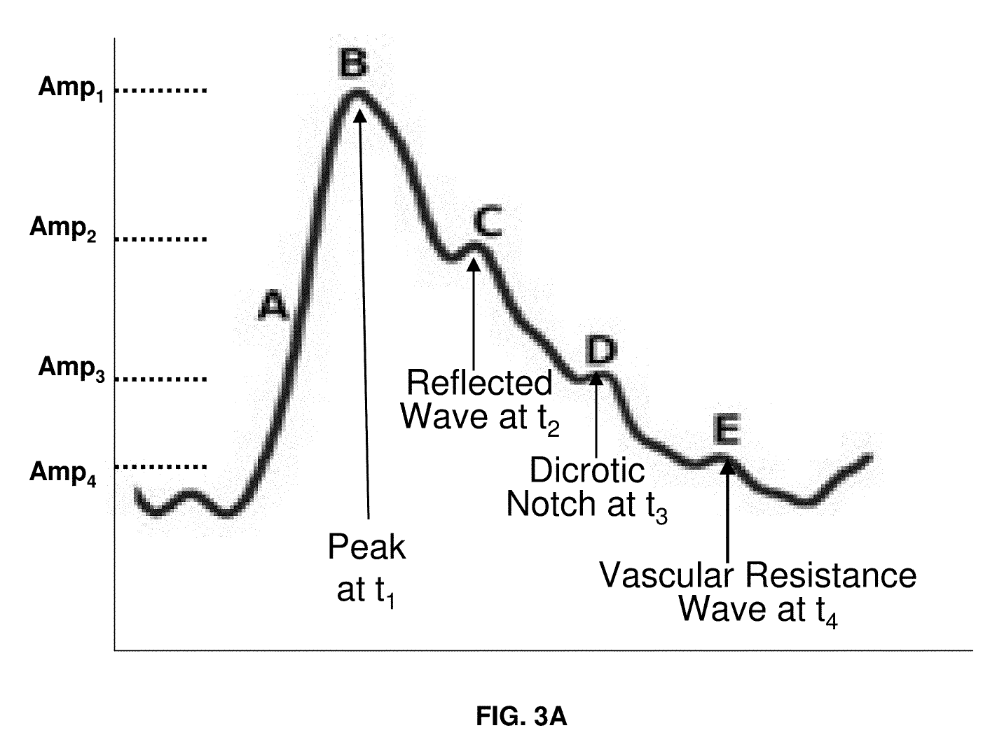

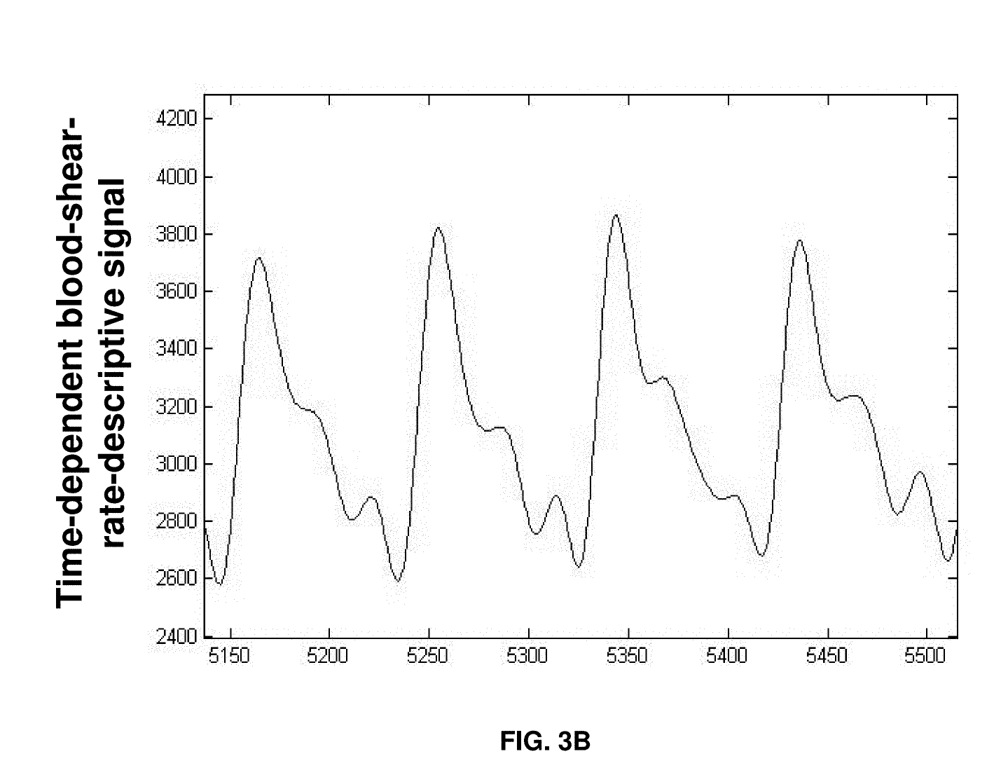

FIG. 3A schematically illustrates a time-dependent blood-shear-rate descriptive DLS signal—FIG. 3B illustrates actual signals that were computed according to presently-disclosed techniques.

Surprisingly, by judiciously processing the scattered-light time-dependent optical response signal using improved DLS signal-analysis techniques, it is indeed possible, using non-invasive optical techniques that probe peripheral blood vessels, to achieve a signal the result FIGS. 3A-3B which preserve various features of the blood pressure waveform not persevered by convention DLS techniques.

As noted above, there is no single and unique transformation function which transforms the scattered-light time-dependent optical response signal into a time-dependent blood-shear-rate descriptive signal.

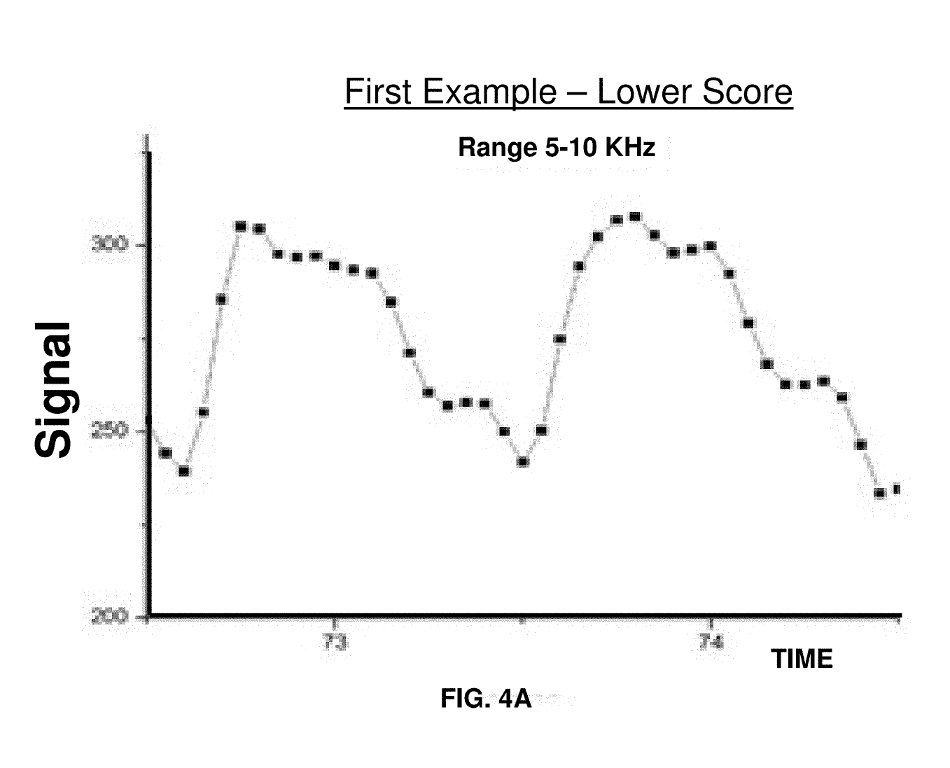

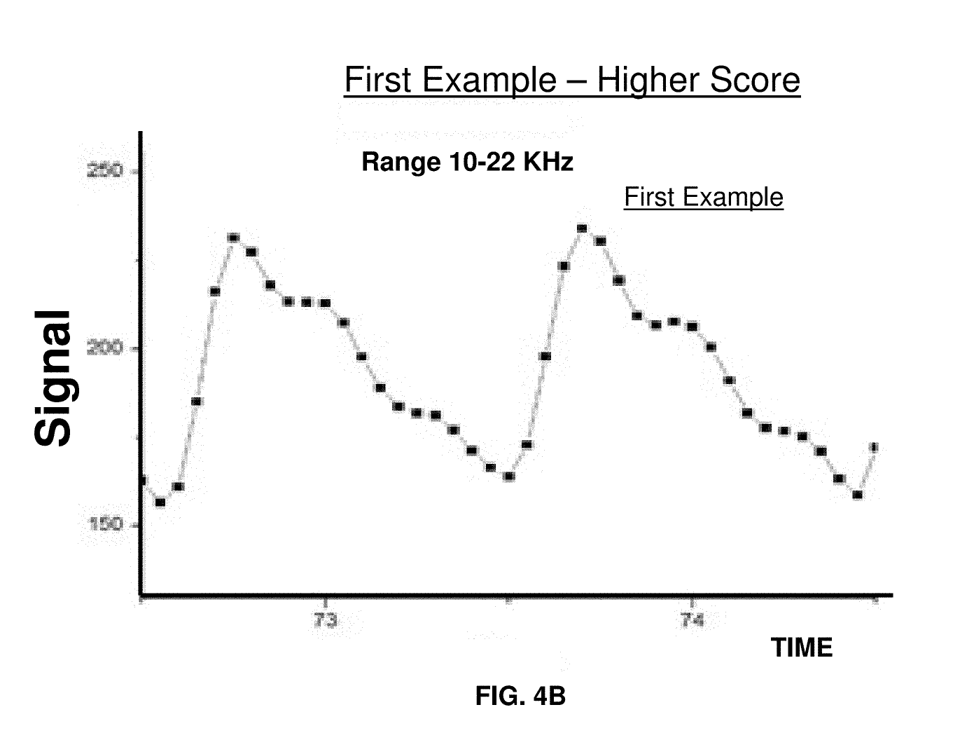

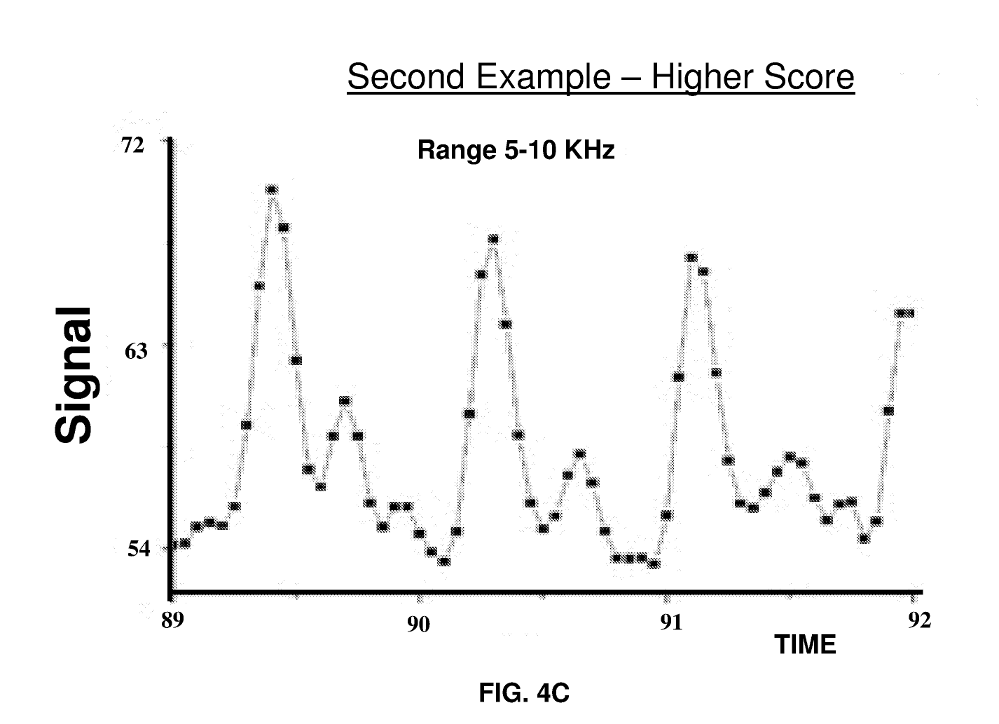

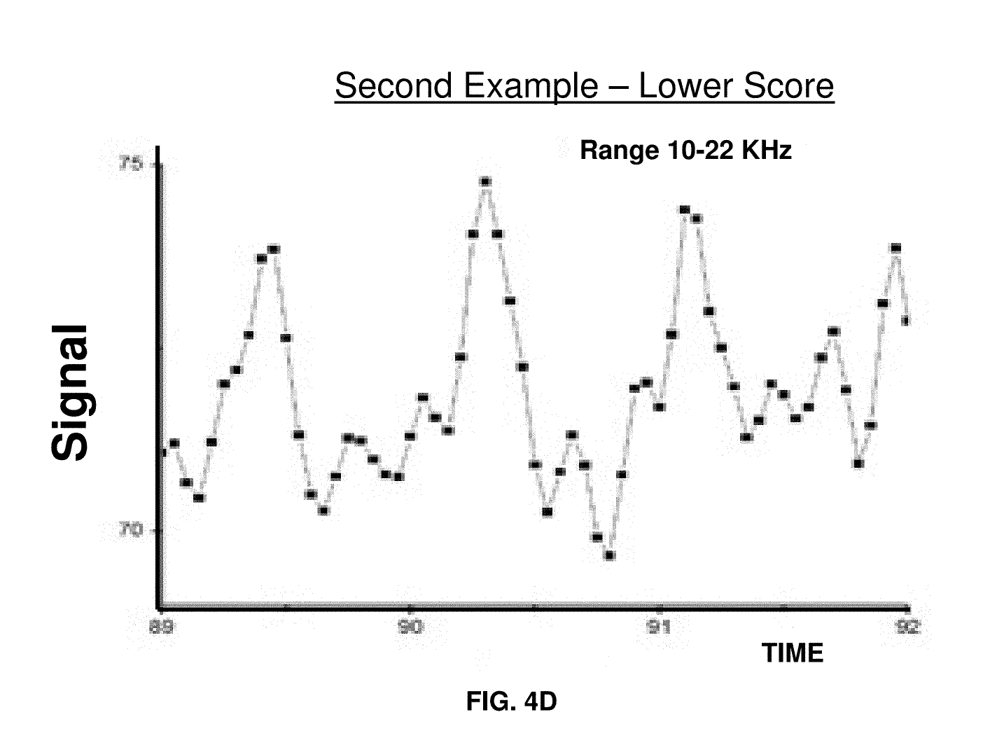

FIGS. 4A-4B illustrate a first example of transforming a scattered-light time-dependent optical response signal into a time-dependent blood-shear-rate descriptive signal. In both cases, when the scattered-light time-dependent optical response signal is processed time-dependent blood-shear-rate descriptive signal, the transformation function (e.g. according to autocorrelation or power-spectrum technique) explicitly or implicitly performs some sort of frequency-selection.

FIGS. 4A-4B relate to the same ‘input’ scattered-light time-dependent optical response signal that is transformed—however, in the example of FIG. 4A, primarily frequencies in the range of 5,000-10,000 KHz were selected/preserved from the scattered-light time-dependent optical response signal, while in the example of FIG. 4B primarily frequencies in the range of 10,000-22,000 KHz were selected/preserved from the scattered-light time-dependent optical response signal.

Inspection of FIGS. 4A-4B and comparison therebetween indicates that the primarily frequencies in the range of 5,000-10,000 KHz were selected/preserved from the scattered-light time-dependent optical response signal more closely resembles the IBP waveform, It is possible to ‘score’ the time-dependent blood-shear-rate descriptive signals of FIG. 4A-4B. In this case, the signal of FIG. 4B has the ‘higher score since it better preserves the waveform features characteristic of the IBP.

FIGS. 4C-4D relate to the same ‘input’ scattered-light time-dependent optical response signal that is transformed—however, the input scattered-light time-dependent optical response signal of FIGS. 4C-4D is different from the input scattered-light time-dependent optical response signal of FIGS. 4A-4B. In the example of FIGS. 4C-4D, the transformation function which primarily selects frequencies of the 5,000-10,000 KHz range (FIG. 4C) generates a time-dependent blood-shear-rate descriptive signal that better preserves waveform features characteristic of the IBP than the transformation function which primarily selects frequencies of the 10,000-22,000 KHz range (FIG. 4D). Thus, FIG. 4C is associated with a ‘higher score’ than FIG. 4D.

Unfortunately, studies performed by the present inventors indicate that it is often unknown a-priori which transformation function (i.e. for processing the scattered-light time-dependent optical response signal into the time-dependent blood-shear-rate descriptive signal) will achieve the time-dependent blood-shear-rate descriptive signal best-preserving the features of the IBP waveform.

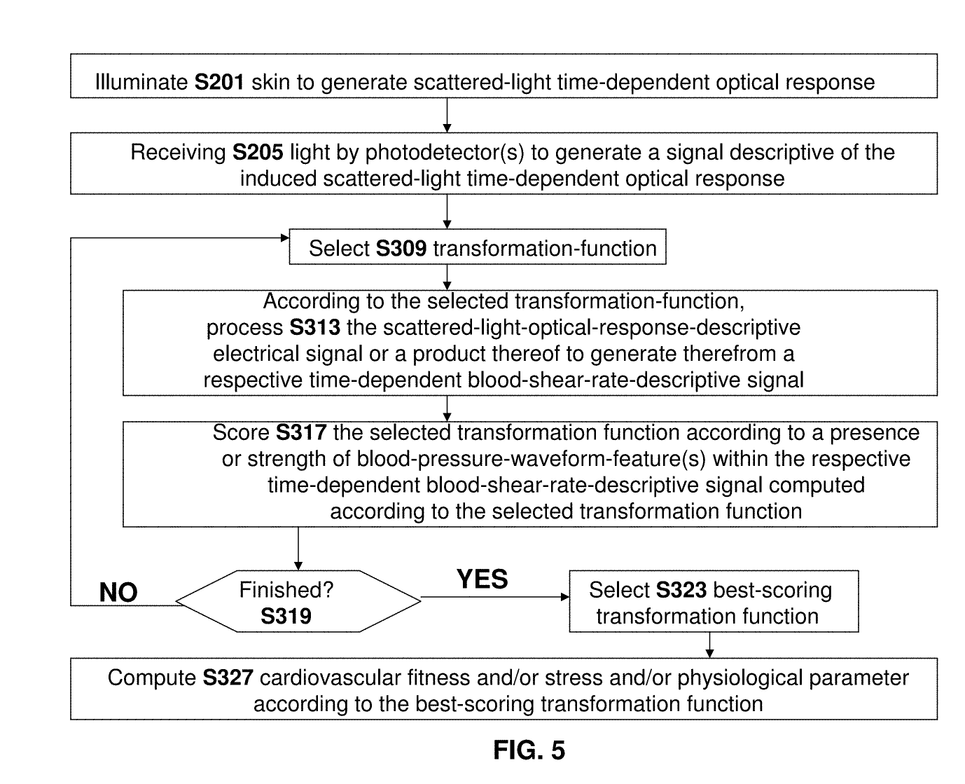

FIG. 5 is a flow-chart of a technique for computing a time-dependent blood-shear-rate descriptive signal and/or a physiological parameter derived therefrom. Steps S201-S205 are as described in FIG. 2B. However, in the method of FIG. 5, there are more than one transformation-functions for processing a scattered-light time-dependent optical response signal into a time-dependent blood-shear-rate descriptive signal—for example, one transformation function primarily selects frequencies in one frequency range (e.g. like in FIGS. 4A and 4C) and another transformation function primarily selects frequencies in another range (e.g. like in FIGS. 4B and 4D),

In step S309 one of the transformation functions is selected—e.g. it is possible to maintain a ‘library’ of transformation functions, and one is selected in step S309. In step S313, the selected transformation function is employed so as to processing the scattered-light time-dependent optical response signal into a respective time-dependent blood-shear-rate descriptive signal, The results are scored in step S317 according to presence of strength of blood-pressure-waveform feature(s)—a list of useful features is set-forth below. Steps S309-S317 are repeated for multiple different transformation-functions until the loop is finished (step S319). The best-scoring transformation function (i.e. selected in step S323 according to the scores) is employed in step S327 to compute the fitness and/or stress and/or physiological parameter.

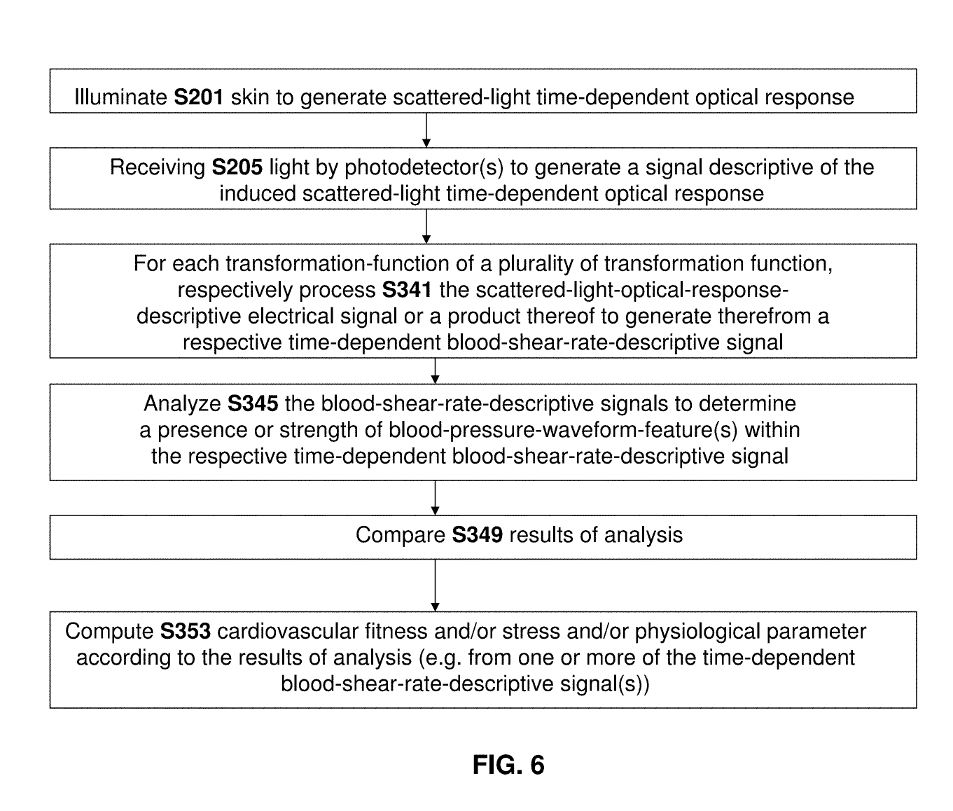

FIG. 6, including steps S201, S205, S341, S345, S349 and S353 illustrates another variations.

Examples of blood-pressure-waveform feature(s) for scoring time-dependent blood-shear-rate descriptive signal include:
(i) a presence, temporal-location, shape or amplitude of a reflected-wave marker (e.g. protrusion) (note that the reflected wave is completely absent from FIG. 1B);
(ii) a presence, temporal-location, shape or amplitude of a vascular resistance wave marker (e.g. protrusion) (note that the vascular resistance wave marker is completely absent from FIG. 1B);
(iii) a presence, temporal-location, shape or amplitude of dicrotic notch;
(iv) a width of the main peak (e.g. illustrated by ‘B’ in FIG. 3B), or of the reflected-wave protrusion/marker, or of the dicrotic notch or of the vascular resistance wave protrusion/marker—or relative measure (e.g. ratio between) any of the aforementioned widths.

Temporal location—this relates to location within the blood-pressure wave-—e.g. determined by any one of (with reference to FIG. 3A)—any tⱼ−tᵢ (where i, j, k, l are positive integers between 1 and 4, j and i are not equal to each other) or any ratio (tₗ−tₖ)/(tⱼ−tᵢ) for any set of i, j, k and l.

Other example of blood-pressure-waveform feature(s) for scoring time-dependent blood-shear-rate descriptive signal include any of the ‘slopes’ (a systolic down-slope, a diastolic down-slope, a systolic upstroke slope)—i.e. absolute values or relative values (e.g. ratios between any two of these 3 slopes).

As noted above, it is not often clear a priori which transformation function yields the same results. Furthermore, even for the same subject, the best-scoring transformation function may fluctuate in time—i.e. for an earlier time-period a first transformation function yields the ‘highest score’ while for a later time-period a second transformation function yields the ‘highest score.’

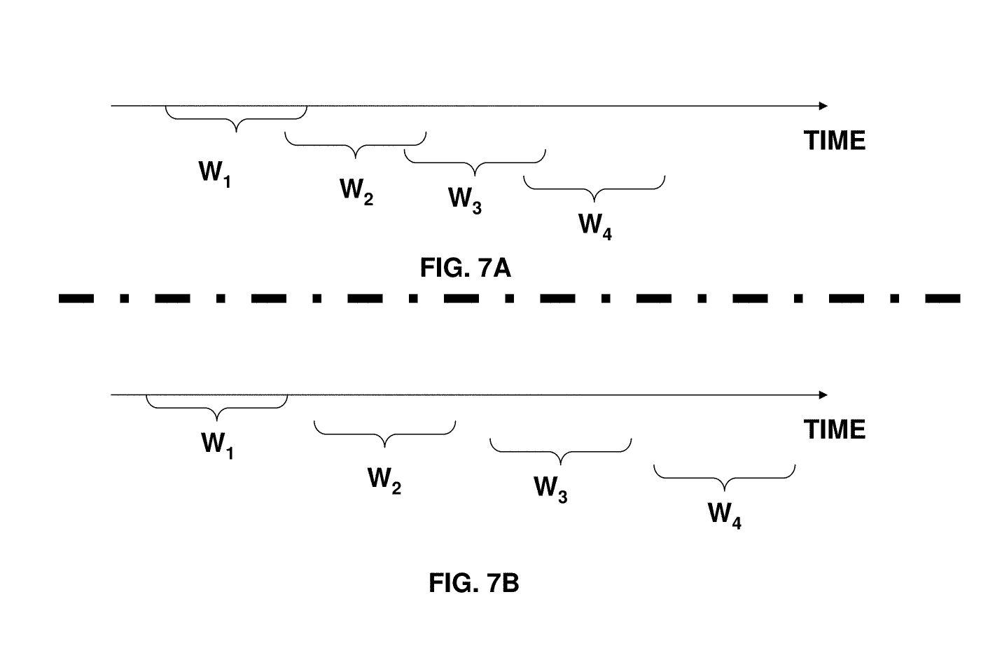

Time periods may be defined according to time windows—see FIG. 7A which illustrates overlapping time-windows and FIG. 7B which illustrates non-overlapping time-windows.

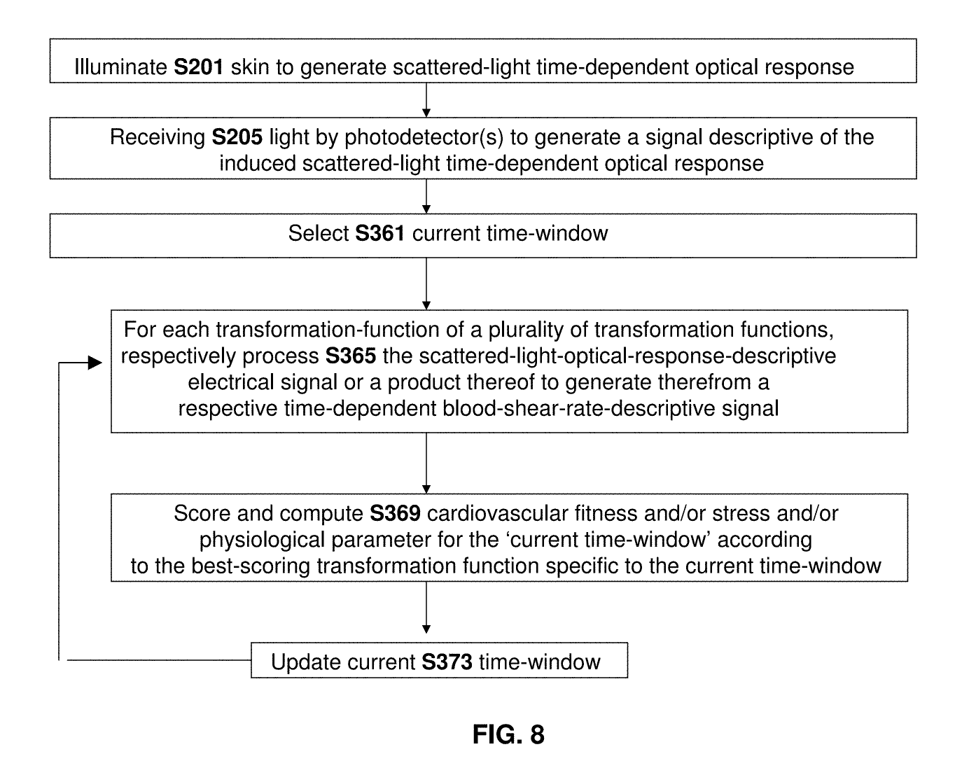

FIG. 8 is an example of a method for processing a scattered-light time-dependent optical response signal into a time-dependent blood-shear-rate descriptive signal according to a dynamic and responsive technique which periodically updates the transformation function (i.e. selected from a ‘family’ of functions) in order to optimize a ‘score’ of the time-dependent blood-shear-rate descriptive signal where the ‘score’ describes resemblance between a time-dependent blood-shear-rate descriptive signal and a blood-pressure waveform.

Thus, in step S361 after a time window is selected, instead of applying only a single transformation function for processing (i.e. for the particular time window) the scattered-light time-dependent optical response signal into a time-dependent blood-shear-rate descriptive signal, it is possible to perform the transformation a number of times—each time, the transformation is performed using a different transformation function. The results are scored in step S369 (e.g. the ‘best-scoring’ time-dependent blood-shear-rate descriptive signal is employed when computing therefrom the cardiovascular fitness and/or stress and/or physiological parameter).

The time window is updated in step S373. For each time window, the ‘best’ transformation function may be different—therefore, the transformation between scattered-light time-dependent optical response signal into a time-dependent blood-shear-rate descriptive signal is said to be performed dynamically in response to scoring for presence and/or strength of features of the blood-pressure waveform.

### Definitions

For convenience, in the context of the description herein, various terms are presented here. To the extent that definitions are provided, explicitly or implicitly, here or elsewhere in this application, such definitions are understood to be consistent with the usage of the defined terms by those of skill in the pertinent art(s). Furthermore, such definitions are to be construed in the broadest possible sense consistent with such usage.

Electronic circuitry may include may include any executable code module (i.e. stored on a computer-readable medium) and/or firmware and/or hardware element(s) including but not limited to field programmable logic array (FPLA) element(s), hard-wired logic element(s), field programmable gate array (FPGA) element(s), and application-specific integrated circuit (ASIC) element(s). Any instruction set architecture may be used including but not limited to reduced instruction set computer (RISC) architecture and/or complex instruction set computer (CISC) architecture. Electronic circuitry may be located in a single location or distributed among a plurality of locations where various circuitry elements may be in wired or wireless electronic communication with each other.

Analog electrical signals or light fields may comprises more than one sub-signal added together in a single electrical (or optical) signal. For example, an analog electrical signal derived from a light field detected by a photodetector that (i.e. where scattered light that is scattered from particles within a fluid contributed to the light field) may be the sum of: (i) a first component (i.e. analog electrical sub-signal) attributable to ambient light (e.g. sunlight); (ii) a second component attributable to skin light-modulating effects; (iii) a third component attributable to regular fluctuations in light intensity due to the presence of a fluorescent bulb and (iv) a fourth component attributable to scattered light that is scattered from particles within a fluid contributed to the light field. Each component or sub-signal of the analog electrical signal is associated with a different respective amount of power.

In some examples, for an analog signal generated by a photodetector, the relative power contribution to overall analog signal power attributable to ambient light is relatively high (i.e. the first component), while the relative power contribution to overall analog signal power attributable to scattered light that is scattered from particles within a fluid is relatively low (i.e. second component).

In general, both a signal and a sub-signal have power levels—the fraction of the power level of the overall signal attributable to a particular portion of the signal or sub-signal is the ‘power fraction’ of the sub-signal or signal component. In the example of the previous paragraph, the power fraction of the overall analog electrical signal due to the ambient light component may be significant (e.g at least 0.1 or at least 0.3 or at least 0.5) while the power fraction of the overall analog electrical signal due to the ‘light scattering’ component (i.e. fourth component) may be relatively low—for example, at most 0.1 or at most 0.05 or at most 0.01).

Embodiments of the present invention relate to generating a ‘hybrid’ signal. A ‘hybrid signal’ derived from a plurality of input analog signals is any non-zero or non-trivial mathematical combination of the input analog signals—i.e. including multiplication, addition, subtraction, etc. The term ‘hybrid’ refers to the fact that the output (or hybrid) signal relates to more than one input signal, and is not restricted to a single input.

Embodiments of the present invention relate to photodetectors (any technology may be used including those listed herein or any other technology). In some embodiments, each photodetector is not infinitesimally small but rather has a size. The ‘distance’ between photodetectors relates to a centroid-centroid distance.

In some embodiments, a light field is comprised of more than on component. Whenever light is generated and reflected or scattered (or modulated in any other manner) to introduce photons into (or to pass through) a certain location (and/or to illuminate the location), this light ‘contributes to’ or ‘influences’ the local light field at that certain location.

Embodiments of the present invention relate to optically measuring a paremter relating to a subject. In different embodiments, this subject is human, or a mammal other than human, or to a warm-blooded animal other than mammals (e.g. birds).

Whenever a power level of a second signal is ‘significantly less’ than a power level of a first signal, a ratio between a power level of the second signal and a power level of the first signal is at most 0.5 or at most 0.3 or at most 0.2 or at most 0.1 or at most 0.05 or at most 0.01.

Some embodiments of the present invention are described for the specific case of only two photodetectors and/or measuring a light field in two locations. The skilled artisan will appreciate that this is not a limitation, any teaching disclosed herein may relate to the case of more than two photodetectors or detecting light fields in more than two locations. Thus, two photodetectors refers to ‘at least two,’ two locations' refers to at least two, and so on.

A stoke-volume may refer to any one of: (i) full-cycle stroke-volume; (ii) partial-cycle stroke volume; (iii) a function describing relative magnitudes (e.g. a ‘radio) between first and second partial-cycle stroke volumes corresponding to first and second portions of the pulse-cycle.

A product of a ‘first signal’ is a second signal that is derived from the first signal—this does not require ‘multiplication.’

A ‘derivative’ of a ‘signal’ is a signal that is derived therefrom—this does not require computing a ‘mathematical derivative’ as is known in calculus.

‘Quantifying a correlation’ between two functions or data-sets refers to computing a slope between the data sets of some of the parameter of curvefitting (linear or non-linear) or a goodness of a fit.

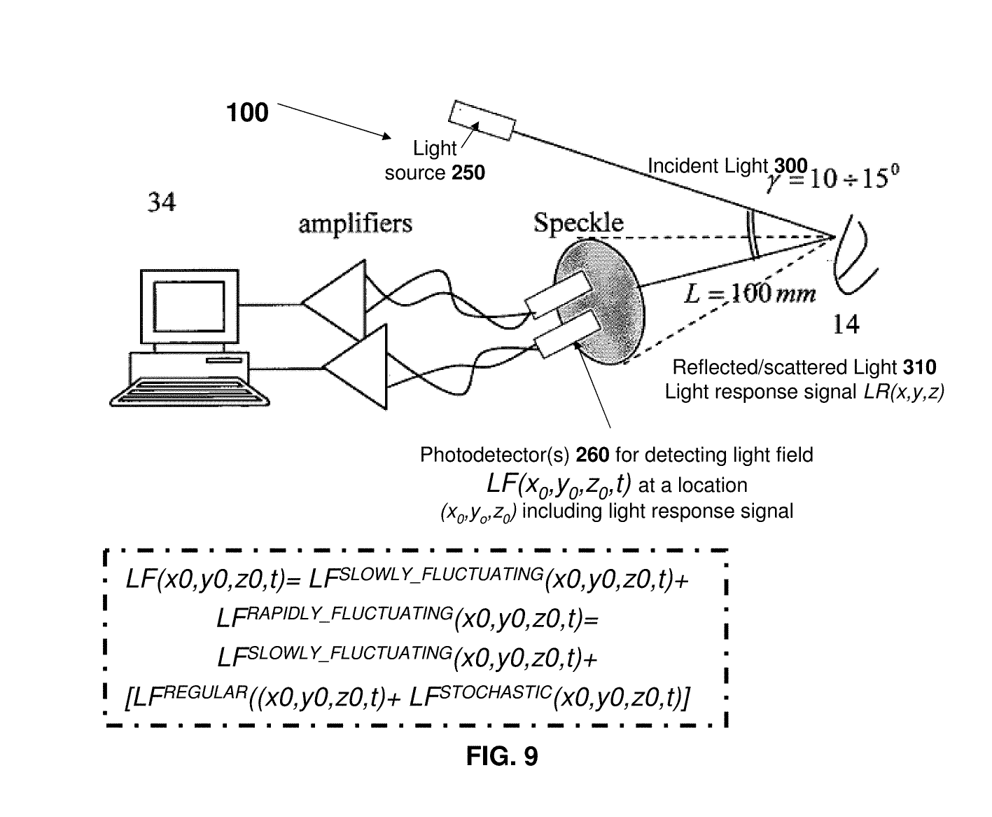

A Discussion of FIG. 9

FIG. 9, reproduced from PCT/US2010/056282, is an illustration of a DLS measurement based system for measuring one or more blood parameters. System 100 includes a light source unit 250 (e.g. laser) for generating at least partially coherent light; optical arrangement (not shown) including focusing optics and possibly also collecting optics; and a detection unit including a photodetector 260. A focused beam of light 300 produced by laser 250 (e.g., a semiconductor laser) is used as a localized light source. In a non-limiting example, a light source unit 250 may be a laser diode (650 nm, 5 mW) or VCSEL (vertical cavity surface emitting laser). The light response i.e. the reflected and/or transmitted light returned from the localized region of the subject's surface 14 (subject's finger in the present example) illuminated with the localized light source 250, can be collected in a determined distance L (in a non-limiting example, L=100 mm) either directly by a detector or via multimode fiber optics. In a non-limiting example, the multimode fiber optics may be a bi-furcated randomized optical fiber where one optical entrance is connected to the detector and another one is optically coupled with the laser diode. In particular, as shown in FIG. 1, system 100 includes at least one laser diode and at least one photodetector (for example, photodiode(s)) appropriately positioned in the reflection-mode measurement set-up.

The photodetector 260 is positioned in space at location (x0,y0,z0) and is configured to detect a light field LF(x₀, y₀, z₀)—i.e. the light field that exists/prevails at point (x₀, y₀, z₀). Typically, the light detected by photodetector 260 comes from a number of sources including but not limited to (A) reflected light 310 which is reflected from and/or scattered by the biological tissue; and (ii) ambient light. Thus, it is possible to write:

LF(x₀,y₀,z₀)=LF_{reflected}(x₀,y₀,z₀)+LF_{ambient}(x₀,y₀,z₀)+other term(s) (EQ. 1)

Throughout the present disclosure, LF denotes a light field.

When light from light source 250 is incident upon biological tissue, (i) a first portion of the incident light is reflected from or scattered from “Brownian particles” (i.e. particles undergoing Brownian motion within a liquid—for example, red blood cells or thromobcytes) to generate a first light response signal whose magnitude/intensity varies stochastically and rapidly in time—this first light response signal is referred to as LF_{reflected}_{_}_{brownian}(x₀, y₀, z₀); (ii) a second portion of the incident light is reflected from stationary biological matter other than Brownian particles—for example, from skin cells, etc—this second portion of the incident light generates a second light response signal whose magnitude/intensity varies at most “slowly” and/or is not stochastic in time—this second light response signal is referred to as LF_{reflected}_{_}ₙₒₙ_{_}_{brownian}(x₀, y₀, z₀);

Thus, it is possible to write:

LF_{reflected}(x₀,y₀,z₀)=LF_{reflected}_{_}ₙₒₙ_{_}_{brownian}(x₀,y₀,z₀)+LF_{reflected}_{_}_{brownian}(x₀,y₀,z₀)+other term(s) (EQ. 2)

In some embodiments, LF_{reflected}_{_}_{brownian}(x₀, y₀, z₀) is indicative of a dynamic light scattering parameter. Unfortunately, in many clinical situations

$$\frac{{LF}_{reflected \_ brownian} ( {x}_{0} , {y}_{0} , {z}_{0} )}{LF ( {x}_{0} , {y}_{0} , {z}_{0} )}$$

and/or

$$\frac{{LF}_{reflected \_ brownian} ( {x}_{0} , {y}_{0} , {z}_{0} )}{{LF}_{reflected \_ non \_ brownian} ( {x}_{0} , {y}_{0} , {z}_{0} )}$$

and/or

$$\frac{{LF}_{reflected\_brownian} ( {x}_{0} , {y}_{0} , {z}_{0} )}{{LF}_{reflected\_ambient} ( {x}_{0} , {y}_{0} , {z}_{0} )}$$

is “small” (for example, less than 0.1 or less than 0.01 or even smaller).
Embodiments of the present invention relate to apparatus and methods for “boosting” the relative contribution to an analog electrical signal of a component indicative of a dynamic light scattering measurement—for example, boosting the relative contribution of an analog electrical signal indicative of LF_{reflected}_{_}_{brownian} (x₀, y₀, z₀).

It is noted that, typically, LF_{ambient} (x₀, y₀, z₀) (see Eqn. 1) and LF_{reflected}_{_}ₙₒₙ_{_}_{brownian}(x₀, y₀, z₀) (see Eqn. 2) have an intensity that is either: (i) “slowly” fluctuating (for example, substantially constant or fluctuating at a rate less than 50 HZ); and/or (ii) “regularly behaved” and non-stochastic. One example of a “rapidly” fluctuating light signal that is regularly behaved and non-stochastic is light from a fluorescent light bulb operating at 50 HZ or 60 HZ. In contrast, the intensity of “speckles pattern light signal” LF_{reflected}_{_}_{brownian} (x₀, y₀, z₀) varies stochastically and rapidly—i.e. at a rate that is at least 50 HZ or at least 100 HZ or at least 200 HZ, depending on diffusion coefficient of the Brownian particle.

Thus, it is possible to write:

$$LF ( {x}_{0} , {y}_{0} , {z}_{0} ) = {LF}_{slowly\_fluctuating} ( {x}_{0} , {y}_{0} , {z}_{0} ) + \underbrace{[ {LF}_{regular} ( {x}_{0} , {y}_{0} , {z}_{0} ) + {LF}_{stochastic} ( {x}_{0} , {y}_{0} , {z}_{0} ) ]}_{rapidly \text{-} fluctuating} + other terms \qquad ( EQ . 3 )$$

where (i) LF_{slowly}_{_}_{fluctuating} (x₀, y₀, z₀) is due to ambient light LF_{ambient} (x₀, y₀, z₀) and/or light reflected from biological tissue other than Brownian particles LF_{reflected}_{_}ₙₒₙ_{_}_{brownian} (x₀, y₀, z₀); (ii) rapidly-fluctuating (i.e. at a rate of greater than 50 HZ and/or 100 HZ and/or 200 HZ) LF_{regular} (x₀, y₀, z₀) is due to ambient light LF_{ambient} (x₀, y₀, z₀); and LF_{stochastic}(x₀, y₀, z₀)=LF_{reeflected}_{_}_{brownian} (x₀, y₀, z₀)/

For the present disclosure, “slowly fluctuating” refers to fluctuations at a rate of less than 50 HZ, while “rapidly fluctuating” refers to regular or stochastic fluctuations at a rate that exceeds 50 HZ (for example, at least 100 HZ or at least 200 HZ).

It is noted that: (i) LF_{stochastic} (x₀, y₀, z₀) is the portion of LF (x₀, y₀, z₀) that may be subjected to DLS analysis to yield one or more blood-related parameters; and (ii) in most clinical situations,

$$\frac{{LF}_{stochastic} ( {x}_{0} , {y}_{0} , {z}_{0} )}{LF ( {x}_{0} , {y}_{0} , {z}_{0} )}$$

is relatively “small” (for example, less than 0.1 or less than 0.01 or even smaller).

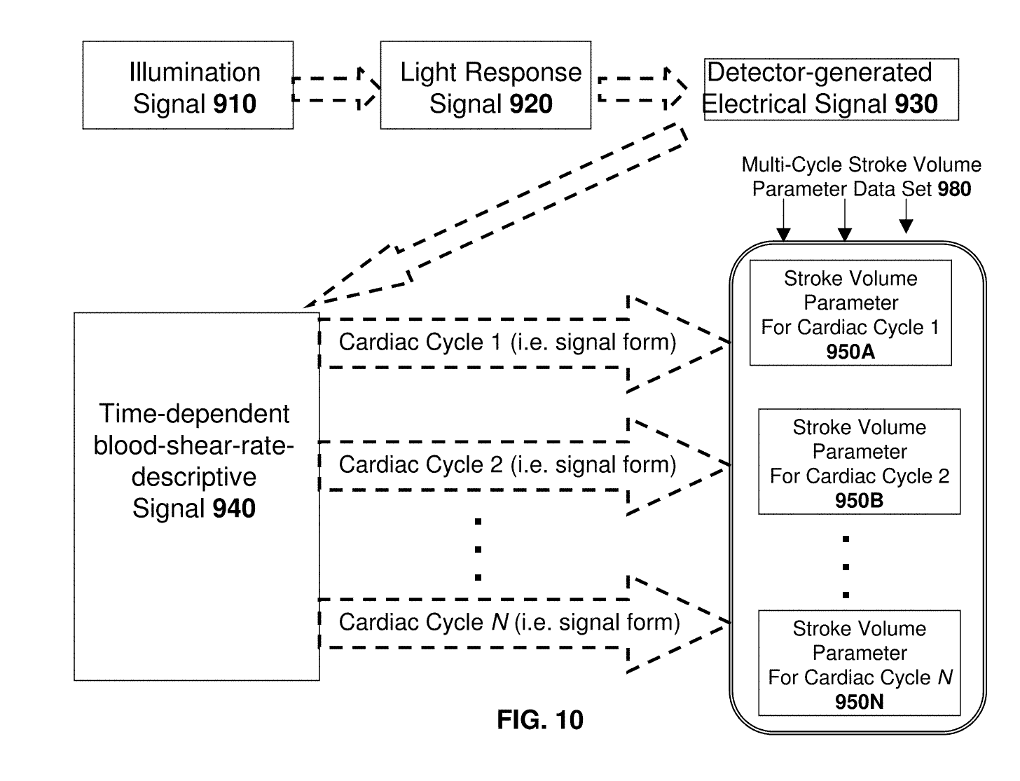

A Discussion of FIG. 10

FIG. 10 is a flow chart of a technique for computing a cardiac-cycle specific stroke volume parameter of a subject.

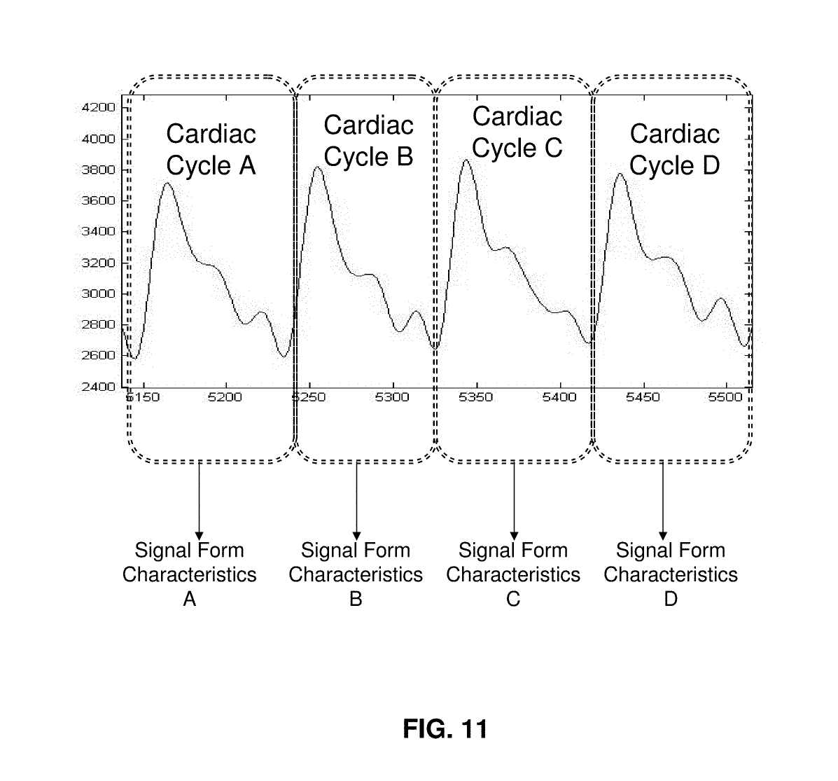

An illumination signal (e.g. from element 10 of FIG. 1A) induces a light response signal 920 (or light field) by reflection and/or transmission and/or deflection by biological tissue. This light response signal is detected by photodectors (e.g. first and second photodetectors) to generate an electrical signal 930 descriptive of light scattering (see FIGS. 10-11). Optionally but preferably, an analog difference signal (e.g. PCT/US2010/056282) is computed.

The analog signal, difference signal or a product thereof (e.g. digitized) is temporally analyzed to compute a blood-shear-parameter signal 940. The time scale of blood-shear-parameter signal 940 (i.e. related to shear—e.g. a rheological parameter which varies in time) is typically much large than the time scale of electrical signal generated by the photodetector which fluctuates very rapidly in time.

As illustrated in FIG. 10, even though this blood-shear-parameter signal fluctuates in time at a much slower rate, its value does, in fact, vary in time, and temporal patterns of this signal may be analyzed. For example, for a plurality of cardiac cycles, signal form parameters may be determined in any manner. According to the signal form parameters, it is possible to compute a stroke volume parameter per cycle—e.g. as disclosed in U.S. patent application 61/884,202 and/or U.S. application 61/884,975 and/or as disclosed in Appendix A).

Collectively, these stroke volume parameters per volume comprise a data set, labeled as 980 of FIG. 10.

This data set may be analyzed to either (i) compute the cardiovascular recovery parameter or (ii) compute the respiratory rate.

FIG. 11 illustrate signal form characteristics—i.e. each cardiac cycle has it's own signal form characteristic—e.g. time of beginning of the cycle, time of end of the cycle, diochroic notch-related times, etc—the skilled artisan is referred to U.S. 61/884,202 and/or U.S. 61/884,975. It is possible to integrate the blood-shear-parameter over time intervals bounded by times of the signal form of the cardiac cycle.

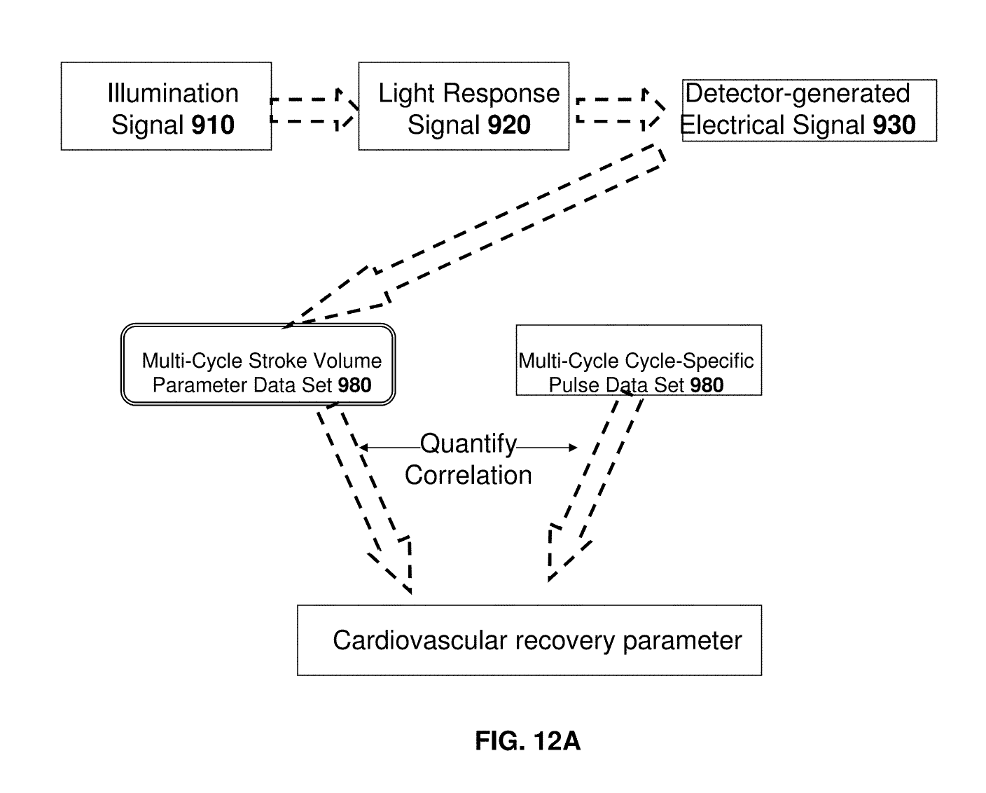

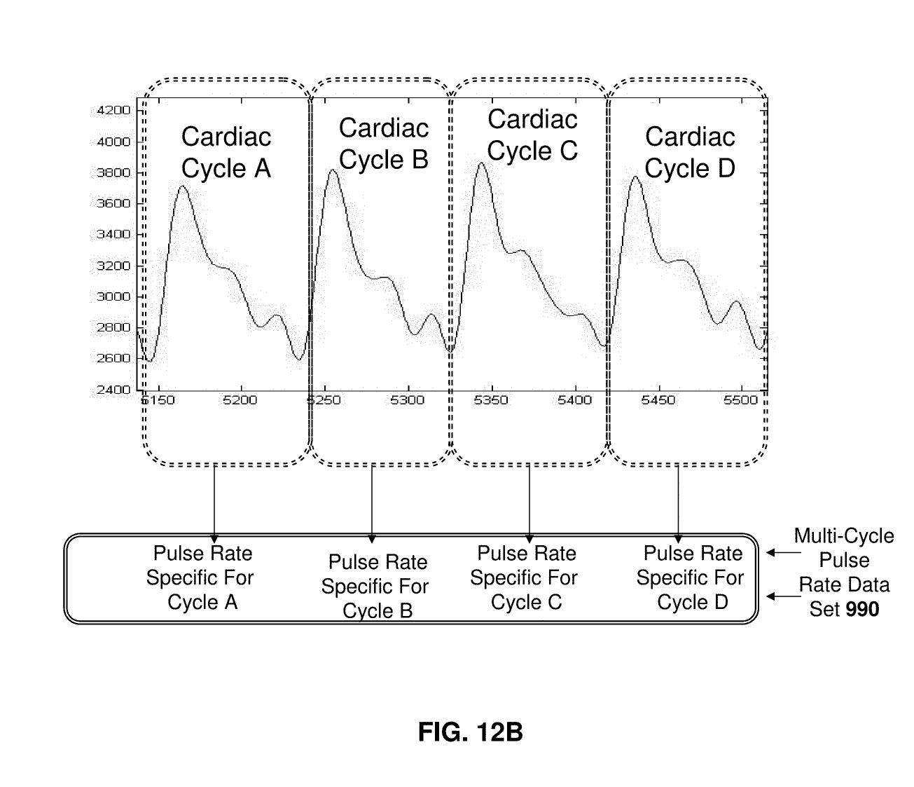

FIGS. 12A-12B relate to correlating between the per-cycle stroke volume parameter and the per-cardiac-cycle pulse rate. As shown in FIG. 12B, the pulse rate is not constant but may fluctuate in time—for each cardiac cycle, it is possible to compute a representative pulse rate.

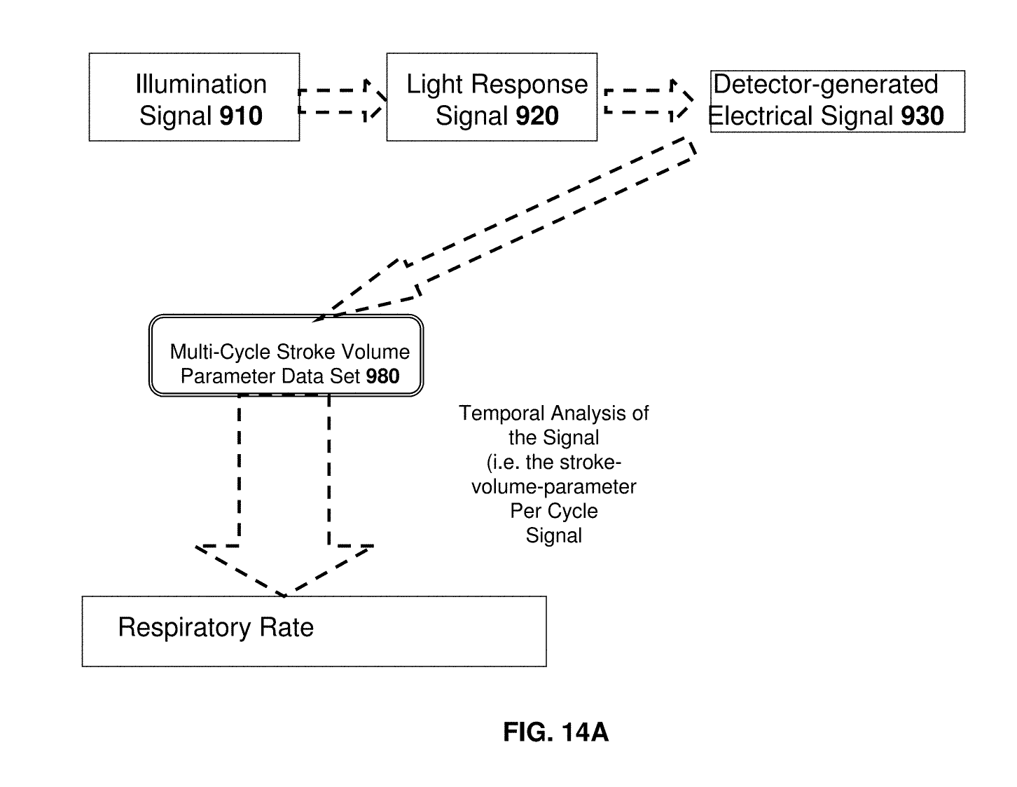

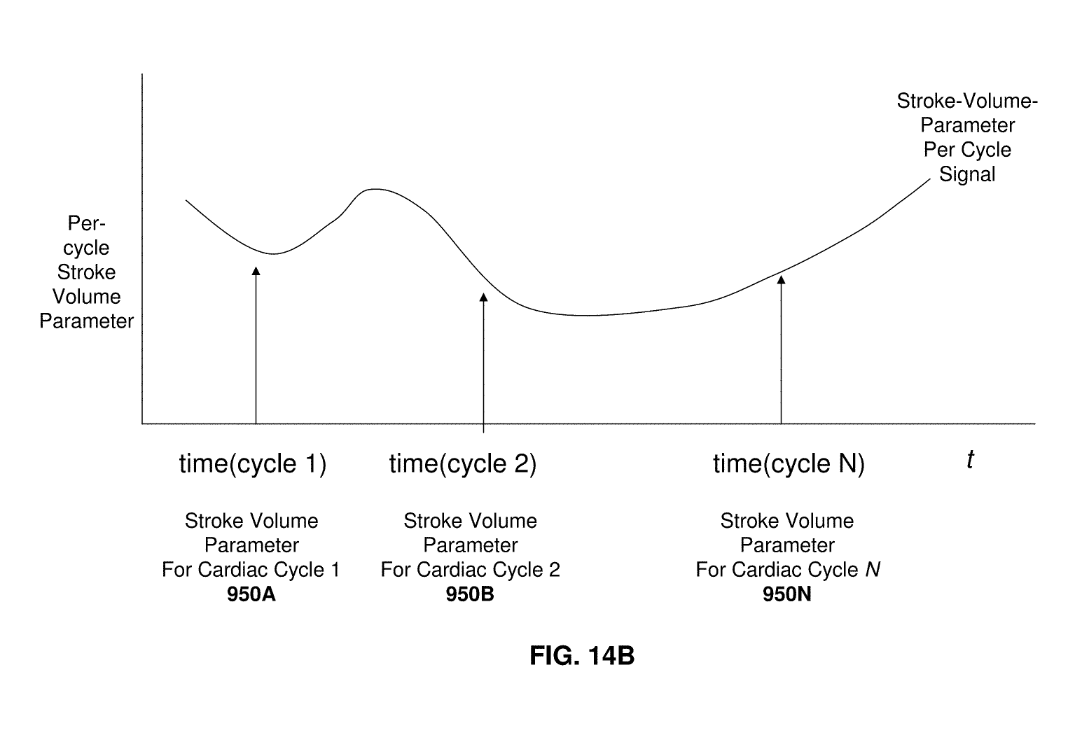

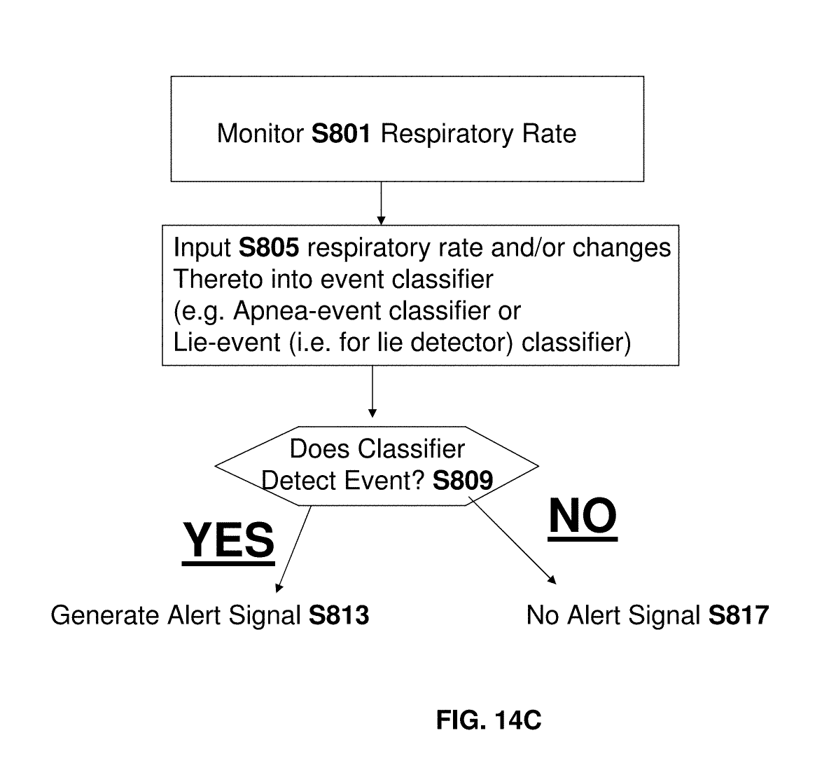

FIGS. 14A-14B relate to a second application of the technique for deriving the per-cycle stroke volume parameter. A respiratory rate may be computed, for example, by analyzing temporal patterns of the signal associated with the per-cycle stroke volume parameter. For example, if the dominant frequency of this per-cycle-stroke-volume parameter is relatively high, this indicates a relatively high respiratory rate. If the dominant frequency of this per-cycle-stroke-volume parameter is relatively low, this indicates a relatively low respiratory rate

In FIG. 14C, this may be employed, for example to detect apnea or lying or any other event associated with respiratory rate. For example, if the subject's pulse drops below a certain threshold (i.e. apnea event) (e.g. for a certain period of time), an alarm signal is generated.

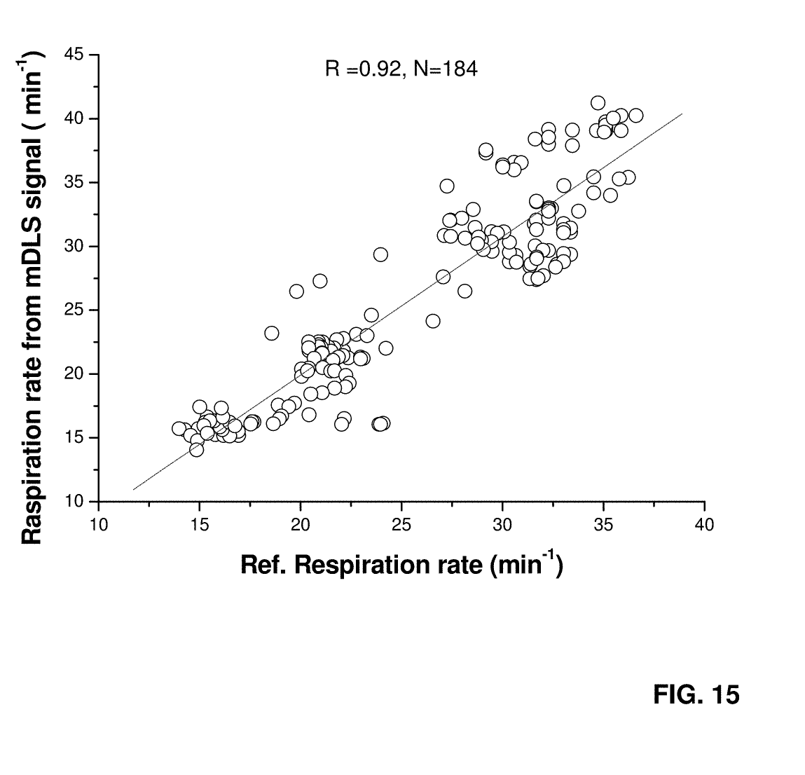

FIGS. 12A-12B and 15 present results when processing actual DLS signals.

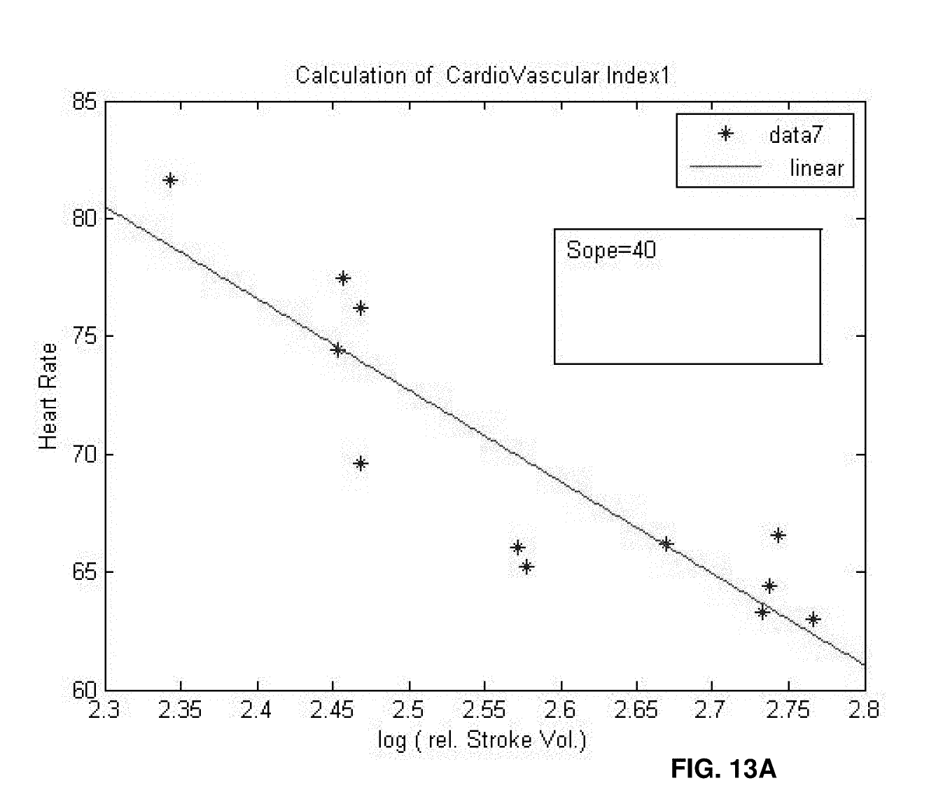

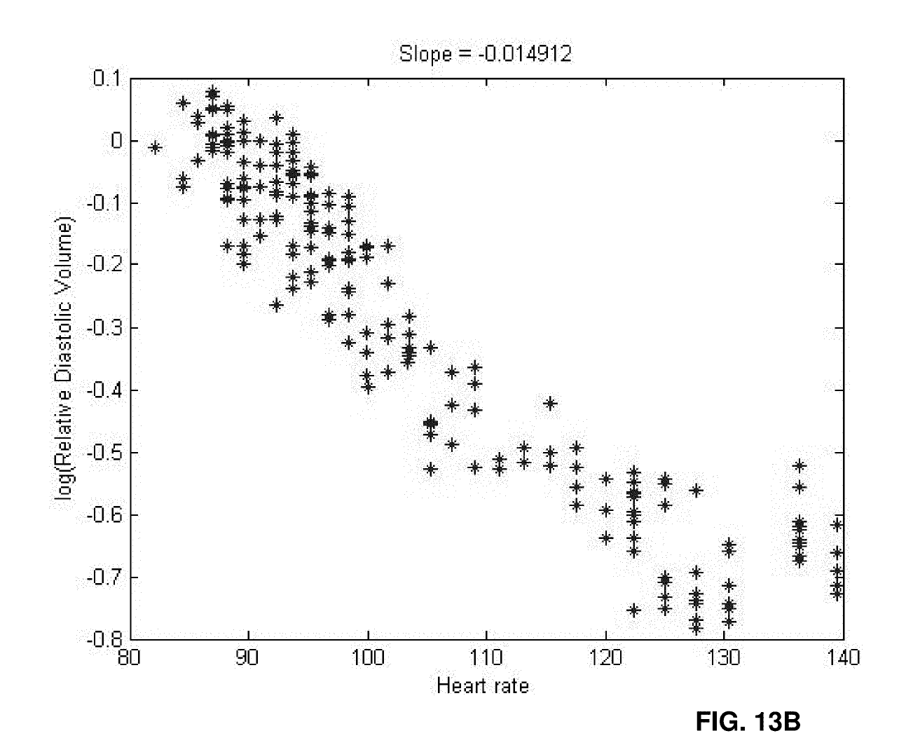

FIG. 12A-12B illustrate computing a correlation between the multi-cycle stroke volume parameter data set 980 (i.e. logs of these values are on the x-axis of FIG. 13A) and cycle-specific heart rate/pulse data set 990 (see the y-axis of FIG. 13A). Preferably this correlation is performed by correlating not the ‘raw value’ of the stroke volume parameter but a logarithm function thereof—this allows one to obtain a linear correlation by using, for example, a linear regression.

FIG. 15 relates to the technique of FIG. 14A.

A method of optically measuring an indication of cardiovascular fitness of a subject comprising:
a. illuminating a target region of the subject by partially or entirely coherent light so as to cause a light response signal from the illuminated region;
b. analyzing quasi-stochastic components and/or component(s) of the light response signal descriptive of distances between scatterers (e.g. blood cells suspended in blood plasma which are scatterers) and/or a component descriptive of Brownian motion in the blood plasma and/or high-frequency components of the light response signal and/or components of the light response signal descriptive of a dynamic light scattering measurement or a photon correlation spectroscopy measurement or quasi-elastic light scattering measurement to compute a blood-rheology and/or blood-shear-related parameter(s) and/or hemodynamic parameter(s);
c. for each cardiac cycle of a plurality of cardiac cycles, subjecting the blood-rheology and/or blood-shear-related parameter(s) and/or hemodynamic parameter to a temporal analysis (e.g. in accordance with the cardiac cycle—e.g. beginning and end-time thereof, commencement of systolic portion thereof, comments of diasolic portion thereof, time of dichrotic notch) within each cardiac cycle to compute therefrom a respective cycle-specific stroke volume parameter;
d. quantifying a correlation (e.g. least squares, goodness-of-fit, slope of the best line), for the multi-cycle set of cardiac cycles defined by the plurality of cardiac cycles, between (i) the cardiac-cycle-specific stroke volume parameter and (ii) the cardiac-cycle-specific pulse rate, (e.g. to derive a heart recovery metric—e.g. recovery from exercise)

In some embodiments, the stroke volume parameter is a parameter of an entire-cycle parameter for entire cardiac cycles.

In some embodiments, the stroke volume parameter is a partial cycle parameter, or a intra-cycle ratio between multiple partial cycle parameters (e.g. between systolic stroke volume and diastolic stroke volume), or an intra-cycle ratio between a partial-cycle parameter and an entire-cycle parameter.

A method of optically measuring an indication of cardiovascular fitness of a subject comprising:
a. illuminating a target region of the subject by partially or entirely coherent light so as to cause a light response signal from the illuminated region;
b. analyzing quasi-stochastic components and/or component(s) of the light response signal descriptive of distances between scatterers (e.g. blood cells suspended in blood plasma which are scatterers) and/or a component descriptive of Brownian motion in the blood plasma and/or high-frequency components of the light response signal and/or components of the light response signal descriptive of a dynamic light scattering measurement or a photon correlation spectroscopy measurement or quasi-elastic light scattering measurement to compute a blood-rheology and/or blood-shear-related parameter(s) and/or hemodynamic parameter(s);
c. for each cardiac cycle of a plurality of cardiac cycles, subjecting the blood-rheology and/or blood-shear-related parameter(s) and/or hemodynamic parameter to a temporal analysis (e.g. in accordance with the cardiac cycle—e.g. beginning and end-time thereof, commencement of systolic portion thereof, comments of diasolic portion thereof, time of dichrotic notch) within each cardiac cycle to compute therefrom a respective first and second cycle-specific stroke volume parameters for each of the cycles
d. quantifying a correlation (e.g. least squares, goodness-of-fit, slope of the best line) the multi-cycle set of cardiac cycles defined by the plurality of cardiac cycles, between (i) the cardiac-cycle-specific stroke volume parameter and (ii) the cardiac-cycle-specific pulse rate, to derive a heart recovery metric.
d. quantifying a correlation, for the multi-cycle set of cardiac cycles defined by the plurality of cardiac cycles, between the first and second cycle parameters are a function of cardiac cycle (e.g. to derive a heart recovery metric—e.g. recovery from exercise)

A method of optically measuring an indication of cardiovascular fitness of a subject comprising:
a. illuminating a target region of the subject by partially or entirely coherent light so as to cause a light response signal from the illuminated region;
b. analyzing quasi-stochastic components and/or component(s) of the light response signal descriptive of distances between scatterers (e.g. blood cells suspended in blood plasma which are scatterers) and/or a component descriptive of Brownian motion in the blood plasma and/or high-frequency components of the light response signal and/or components of the light response signal descriptive of a dynamic light scattering measurement or a photon correlation spectroscopy measurement or quasi-elastic light scattering measurement to compute a blood-rheology and/or blood-shear-related parameter(s) and/or hemodynamic parameter(s);
c. for each cardiac cycle of a plurality of cardiac cycles, respectively analyzing the blood-rheology and/or blood-shear-related parameter(s) over time within each cardiac cycle to compute therefrom a respective first and second cycle-specific stroke volume parameters for each of the cycles;
d. quantifying a correlation, for the multi-cycle set of cardiac cycles defined by the plurality of cardiac cycles, between the first and second cycle parameters are a function of cardiac cycle (e.g. to derive a heart recovery metric—e.g. recovery from exercise)

A method of monitoring heart-health of a subject, said system comprising:
a. illuminating a target region of the subject by partially or entirely coherent light so as to cause a light response signal from the illuminated region;
b. analyzing quasi-stochastic components and/or component(s) of the light response signal descriptive of distances between scatterers (e.g. blood cells suspended in blood plasma which are scatterers) and/or a component descriptive of Brownian motion in the blood plasma and/or high-frequency components of the light response signal and/or components of the light response signal descriptive of a dynamic light scattering measurement or a photon correlation spectroscopy measurement or quasi-elastic light scattering measurement to compute a blood-rheology and/or blood-shear-related parameter(s) and/or hemodynamic parameter(s);
c. monitoring a blood-shear-related and/or rheology-related parameter PARAM(BLOOD) over time by measuring the blood-shear-related and/or rheology-related parameters for a plurality of times {t1, t2, . . . tN} to compute respective values {PARAM(BLOOD)ₜ₁, PARAM(BLOOD)ₜ₂, . . . PARAM(BLOOD)_{tN}} of the blood parameter of each of the plurality of time {t1, t2, . . . tN} (e.g each data point of FIG. 6A), each respective parameter-computing of PARAM(BLOOD)ₜᵢ comprising respectively analyzing respective temporal parraerns of a quasi-stochastic component of a light signal response signal responsive to the illumination of the target region, the light response signal being descriptive of speckle patterns over a respective time period historical to historical to the time ti, of the blood-shear-related and/or rheology-related parameter PARAM(BLOOD) to march forward in time;
c. deriving a heart stroke-volume and/or heart-recovery parameter from the blood-shear-related and/or rheology-related parameters (e.g. minimum time interval) by computing respective time-integration-functions INT_{FIRST} and INT_{SECOND} thereof over first and second time-integration intervals [T_{A} . . . T_{B}] and [T_{C} . . . T_{D}] and by computing the heart stroke-volume and/or heart-recovery parameter from a ratio between the time-integration-functions INT_{FIRST} and INT_{SECOND}.

A device for optically measuring one or more blood parameters of an adult human user:
a. a device housing having a rigid inner arm-facing surface facing towards the user's arm when worn and an outer display surface facing away from the user's arm when worn, the arm-facing surface closely matching a skin surface over the user's radius to substantially cover, within a tolerance of at most 1 mm, a continuous skin surface portion having an axial length of 1 cm and subtending an angle of at least 30 degrees;
b. one or more light sources mounted to the device housing configured, when worn, to illuminate the covered skin surface and/or tissues (i.e. locations of the radius) below the covered skin surface;
c. one or more light detectors configured to detected reflected light; and
electronic circuitry.

A method of optically measuring a cardiovascular parameter of a subject, the method comprising:
a. optically measuring at least blood-velocity-spatial-variation parameter selected from the group consisting of:
i. a representative (e.g. an average or a waited average) blood shear rate within the illuminated region or within a portion thereof;
ii. a representative (e.g. an average or a waited average) blood velocity spatial derivative (or variation of blood velocity) within the illuminated region or within a portion thereof;
b. in accordance with the optically measured blood-velocity-spatial-variation parameter, computing at least one of:
i. a heart stroke volume parameter (e.g. a heart stroke volume or a cardiac output, or an ejection fraction);
ii. an extent of a dependence of the heart stroke volume parameter upon heart rate; and/or
iii. an extent of a dependence of a first one of the heart stroke volume parameters upon a second one of the heart stroke parameters.

A method of optically measuring a cardiovascular parameter of a subject, the method comprising:
a. illuminating a portion of the subject's skin by partially or entirely-coherent to generate a coherent light-interference pattern by scattering the partially or entirey-coherent light off of moving red blood cells (RBCs) within or beneath the subject's skin;
b. analyzing the coherent-light interference pattern (e.g. time fluctuations in the pattern) derived from the light scattering off of the RBCs to compute a time-fluctuating blood-velocity-spatial-variation parameter;
c. in accordance with the results of the analysis of the coherent-light interference pattern, computing at least one of:
i. a heart stroke volume parameter (e.g. a heart stroke volume or a cardiac output, or an ejection fraction);
ii. an extent of a dependence of the heart stroke volume parameter upon heart rate; and/or
iii. an extent of a dependence of a first one of the heart stroke volume parameters upon a second one of the heart stroke parameters.

A method of optically measuring a cardiovascular parameter of a subject, the method comprising:
a. illuminating a portion of the subject's skin by partially or entirely-coherent to generate a coherent light-interference pattern by scattering the partially or entirey-coherent light off of moving red blood cells (RBCs) within or beneath the subject's skin;
b. analyzing the coherent-light interference pattern (e.g. time fluctuations in the pattern) derived from the light scattering off of the RBCs to compute at least blood-velocity-spatial-variation parameter selected from the group consisting of:
i. a representative (e.g. an average or a waited average) blood shear rate within the illuminated region or within a portion thereof;
ii. a representative (e.g. an average or a waited average) blood velocity spatial derivative (or variation of blood velocity) within the illuminated region or within a portion thereof;
c. time-integrating the blood-velocity variation parameter over a time interval whose temporal boundaries are selected according to a cardiac-cycle of the subject.

In some embodiments, one or both of the temporal boundaries are either: (i) an initiation of a systole phase; (ii) an initiation of a diastole phase.

In some embodiments, the temporal boundaries correspond to a single cardiac cylcle.

In some embodiments, the temporal boundaries correspond to a portion of a cardiac cycle (e.g. systolic or diastolic).

In some embodiments, the time-integrating is performed first and second times for respective sets of temporal boundaries, and a ratio between the results of the first and second time-integrating is computed.

An apparatus comprising: a source of partially or entirely-coherent light;

a photodetector assembly; and electric circuitry, the apparatus configured to perform any method disclosed herein.

It is further noted that any of the embodiments described above may further include receiving, sending or storing instructions and/or data that implement the operations described above in conjunction with the figures upon a computer readable medium. Generally speaking, a computer readable medium may include storage media or memory media such as magnetic or flash or optical media, e.g. disk or CD-ROM, volatile or non-volatile media such as RAM, ROM, etc. as well as transmission media or signals such as electrical, electromagnetic or digital signals conveyed via a communication medium such as network and/or wireless links.

Having thus described the foregoing exemplary embodiments it will be apparent to those skilled in the art that various equivalents, alterations, modifications, and improvements thereof are possible without departing from the scope and spirit of the claims as hereafter recited. In particular, different embodiments may include combinations of features other than those described herein. Accordingly, the claims are not limited to the foregoing discussion.

### APPENDIX A

Embodiments of the present invention relate to techniques for computing (i) heart stroke volume (e.g. per cycle or for a portion thereof); (ii) heart parameters related to post-exercise recovery or heart recovery rate (HRR); (ii) cardiovascular fitness level by computing a time integral of the blood shear and/or blood-rheology parameter as derived from DLS measurements over time intervals whose initial times and ending times correlate to the cardiac cycle—e.g. as computed by the blood shear and/or blood-rheology parameter (e.g. see FIG. 6).

Thus, it is possible to compute a time-integral of the blood-shear-rate-descriptive signal F(T) [F(T) may be a blood-shear-rate parameter or another parameter descriptive spatial fluctuations of blood velocity (e.g. a spatial derivative of blood velocity))]

V=∫_{T1}^{T2}F(t)*A*dt (EQ 1)

and to select T1 and/or T2 to correlate with meaningful times of the cardiac cycle—.eg. the beginning of the systolic portion of the cycle, beginning of the diasystolic portion of the cycle, the dicrotic notch or any other time of the cardiac cycle.

In the time interval [T1,T2] of EQ1, T1 and T2 are ‘ temporal boundaries’ oif the time integral.

The limits of the integral (i.e. or other time-analysis parameter) of EQ 1 are selected by analyzing the signal F(T) or any other indication to correlate F(t) with a cardiac cycle and/or pulse wave form.

For the present disclosure, a ‘cardiac cycle’ and a pulse wave (or pulse wave form) are used interchangably—the time

The integral of EQ 1 one example of a “cardiac-cycle-specific stroke volume parameter’—i.e. it describes heart stroke volume for a specific cardiac cycle or portion thereof.

In some embodiments, it is possible to compute a ratio between such integrals or a ratio between ‘cardiac-cycle’-specific stroke parameters (e.g. where each parameter is derived from temporal analysis of the rheological and/or

‘—for example,

$$\frac{{\int }_{{V}_{1}}^{{V}_{2}} F ( t ) * A * dt}{{\int }_{{V}_{3}}^{{V}_{4}} F ( t ) * A * dt}$$

—for example, T1 may be the beginning of the systolic portion, T2 may be the end of the systolic portion, T3 may be the beginning of the diastolic portion, T4 may be the end of the diastolic portion.

Furthermore, in the event that the time-bounds of the integral of EQ(1) are within a single cardiac cycle, the value computed in EQ(1) may vary over cardiac cycles—i.e. each cardiac cycle may produce a different value.

It may be possible to correlate a value of V from EQ(1) with another property of the cardiac cycle to which it relates. For example, the subject's pulse may change over time—e.g. after the subject completes an exercise regime and begins to rest, his/her pulse may decrease as he/she recovers. Each cardiac cycle may be associated with a different ‘insanities pulse value’.

As discussed above it is possible to correlate V from EQ(1) with the instantaneous pulse rate—e.g. the slope of FIG. 13A-13B may be indicative of the heart recovery time or the general heart health.

Thus, in FIG. 13A-13B each datapoint (illustrated as a star) describes for a specific cardiac cycle (i) the heart rate on the y-axis (i.e. the heart rate for a specific cardiac cycle) and (ii) a stroke volume parameter for that cycle as computed by temporal analysis of the blood rheology and/blood-rheology and/or blood-shear-related parameter(s) and/or hemodynamic parameter (i.e. in accordance with times that correlate with a cardiac cycle).

The data of FIGS. 13A-13B relates to multiple cardiac cycles—each data point is from another cardiac cycle, and each data point is cycle-specific (i.e. relating to an entirety of a cardiac cycle or to a portion thereof—e.g. systolic portion or diastolic portion).

FIG. 13A-13B illustrates the quantifying a correlation between cycle-specific heart rate (y axis) and cycle-specific stroke volume parameter. In the case of FIG. 13A-13B, the quantification of the correlation may be a description of the ‘goodness of fit’ for a least squares routine or may be a slope of the best line (e.g. or another pre-determined fiting function).

In FIG. 13A, there are 12 datapoints, and thus the the multi-cycle set of cardiac cycles is 12 cardiac cycles.

Referring to FIG. 13B, the heart of a healthy-person (i.e. an athlete) of may operate efficiently both at high rates (i.e. during exercise) and at low rates (i.e. during rest). In contrast, an the heart of an unhealthy person may operate much more efficiently at rest than during exercise. In the example of FIG. 8, we would expect the slope for the healthy person/athlete to be much more shallow—i.e. the heart efficiency parameter (i.e. x-axis) is not so dependent upon the heart rate.

In the example of FIG. 13, the stroke volume parameter is the relative stroke volume for only the diastolic portion of the cardiac cycle.

### APPENDIX B

### Measurement of Hemodynamic Response by Using Shear Rate Related Dynamic Light Scattering Sensor.

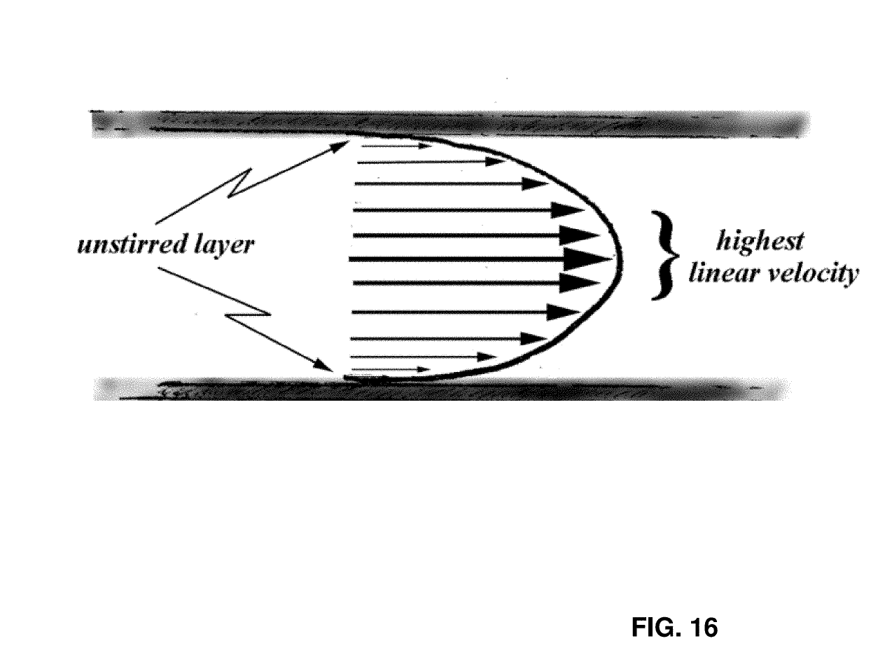

The mDLS pulsatile signal is originated from the relative movement of the scattering particles. In the case of the blood flow the relative movement is caused by the velocity profile of the flowing blood in small vessels (see FIG. 16).

The proposed mDLS technology takes adventage of the RBC velocity differences and produces a signals that resemble other well knows physiological signals such as PPG, Laser Doppler Velocimetry (LDV), and Invasive Blood Pressure (IBP). mDLS differs from the LDV in that LDV measures the local velocity of blood flow, whereas the mDLS measures the red blood cells (RBC) velocity gradient which is directly related to the shear rate. According to basic low of laminar flow the shear rate increases when the velocity goes up, so the mDLS signal is a function of flow velocity.

In case of mDLS the measured signal is formed by the difference in Doppler shifts of all correlated and uncorrelated particles in the scattering volume. The signal is derived from the fluctuations of the intensity signal I(t). The measured parameter is the autocorrelation function of I(t) which is defined by:

g(τ)= [symbol] I(t)·I(t+τ) [symbol] − [symbol] I [symbol] ²

This parameter can be expressed in terms of decay time

g(τ)≈exp(−τ/τ₀)=exp(−Γ·τ)

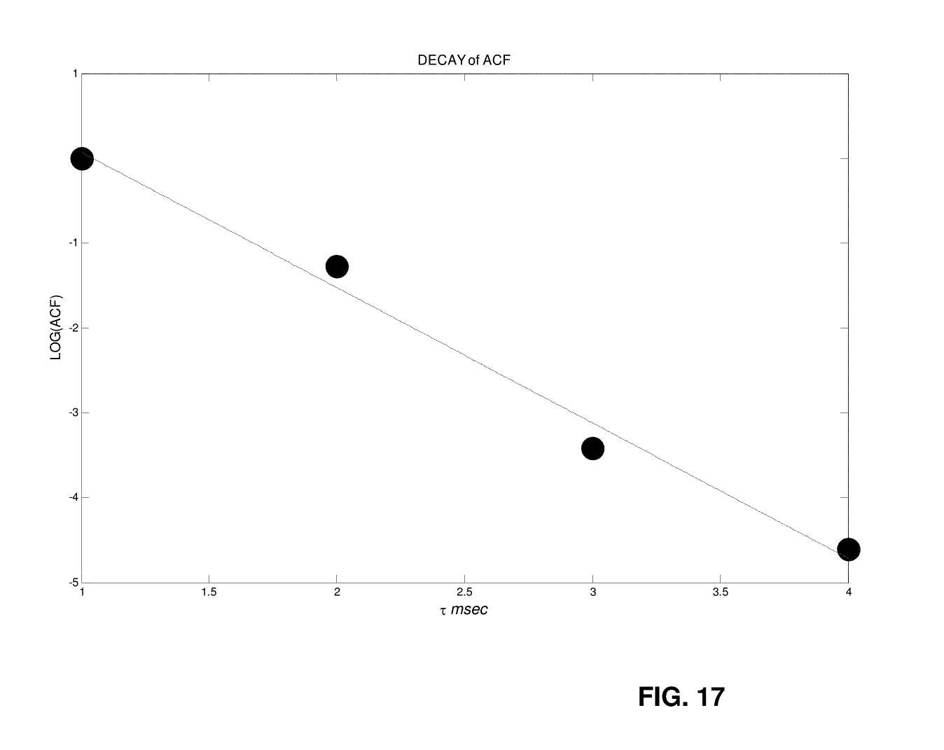

Example below demonstrated how the ACF looks like for the pulsatile signal measured by using mDLS (FIG. 17)

An alternative method to characterize the DLS signal is to express it in terms of power spectrum.

According to the Wiener-Chintschin theorem the equivalent representation of the autocorrelation function exp(−Γ·τ) in terms of power spectrum can be given by the Lorentzian function:

$$P ( ω ) = \frac{2 \cdot Γ}{{Γ}^{2} + {ω}^{2}} ,$$

One of the essentially required features of our in-vivo measurement system is the ability to reject strong motion artifacts, which may come into appearance at very low frequencies of the spectrum. Moreover, strong fluctuations of the highly energetic low-frequency components can lead to the saturation of the measured signal and, subsequently, reduce the dynamic range of the measurement system.

This problem which is essential for in-vivo measurement can be solved by using the analog subtraction method. Such a filter changes the measured characteristics of the power spectrum and the explicit response of the filter has to be taken into consideration. In our system the first-order analog filter was used.

If the frequency response of this filter is characterized by

$$ξ ( ω ) = \frac{χ \cdot ω}{\sqrt{1 + {( χ \cdot ω )}^{2}}}$$

Then the highest signal-to-noise ratio is achieved for the integral value of the entire spectrum:

$$S ( Γ ) = {\int }_{0}^{∞} \frac{2 \cdot Γ}{{Γ}^{2} + {ω}^{2}} \cdot ζ ( ω ) \cdot d ω ,$$

By defining Φ=χ·Γ we get

$$S ( Γ ) = \frac{2 \cdot Φ \cdot [ arctan ( \frac{1}{\sqrt{{Φ}^{2} - 1}} ) - π ]}{\sqrt{{Φ}^{2} - 1}} ,$$

So for any given χ and S the value of Γ can be calculated. The major advantage of using such integral characteristic as S is its superiority over Γ, in terms of signal to noise ratio. In case that the sought Γ is located far from the cut-off point of the filter, the Γ value is slightly affected by its characteristics (11). Thus, the results of in-vivo measurement can be expressed either in terms of S or directly in terms of Γ.

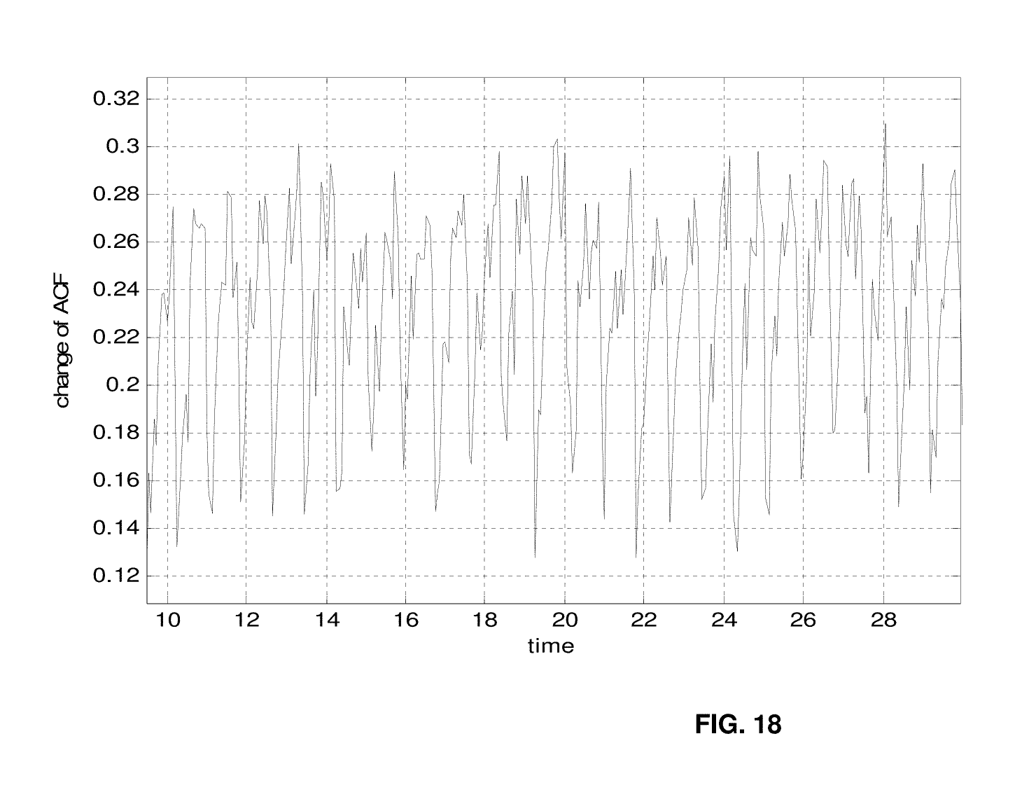

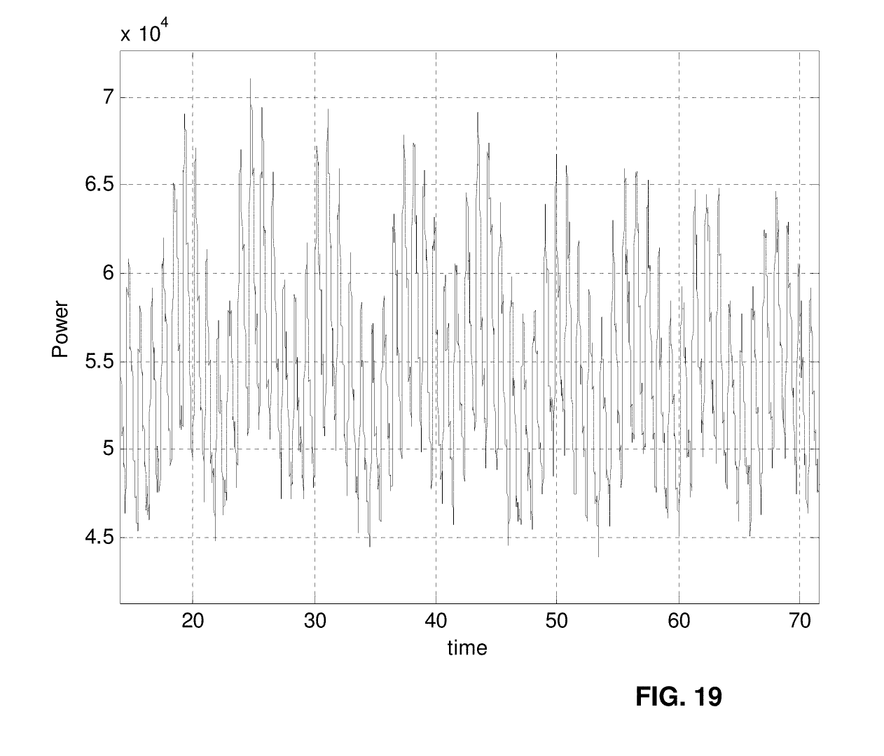

On the figure below example demonstrating the typical change of autocorrelation function (ACF) of the DLS signal being measured from the finger tip (FIG. 18)

The pulsewave is clearly followed by this dimensionless parameter in terms of ACF. It's value reflects the changes of the shear rate of the flow.

While the ACF enables to make an assessment of the blood velocity which is independent on a number of scatterers the spectral energy characteristics are dependent on both the number of RBC's and their shear rate and therefore more related to local blood flow.

Here is an example of mDLS pulse wave measurements in terms of power spectral characteristics or S. We can observe low frequency fluctuations associated with a blood pressure modulations which are associated with the sympathetic nervous system activity (Traube wave) (FIG. 19)

### Rheological Considerations:

When we are talking about ensemble of moving particles than the measured signal is a weighted sum of all elements of the ensemble. For the laminar blood flow the most important contribution to the correlation function measured by mDLS comes from all moving RBC pairs. These pairs are formed by the spatially related moving RBC's, which are located in close vicinity to each other. The more distant particles give negligible weight into the g. Therefore, the mDLS is sensitive to the velocity gradient in laminar or turbulent flow. The velocity gradient is originated from the blood pressure gradient.

In the case of Poiseuille laminar blood flow the blood moves back and forth with oscillatory frequency ω in response to the oscillatory pressure gradient. The flow velocity u(r,t) is the function of radial location r in the vessel and time t. u(r,t) is described by:

$$u ( r , t ) = i \frac{{k}_{s} {a}^{2}}{μ \cdot {Ω}^{2}} ( 1 - \frac{{J}_{0} ( ζ )}{{J}_{0} ( Λ )} ) \cdot {e}^{iwt}$$

Where J₀ is Bessel function of order zero,

$$Ω = \sqrt{\frac{ρω}{μ} a} ,$$

“a” is the vessel radius and ρ is density. Additionally,

$$Λ = ( \frac{i - 1}{\sqrt{2}} ) \cdot Ω ,$$

ξ(r)=Λ·r/a, kₛ is amplitude of pressure gradient, and μ is the coefficient of viscosity. The signal of mDLS will be determined by the relative velocity of the paired RBC particles, or the value of

$$\frac{\partial ( u ( r , t ) )}{\partial r} .$$

It can be shown that

$$\frac{\partial ( u ( r , t ) )}{\partial r} \approx - \frac{{k}_{s} \cdot a}{Λ} \cdot ( \frac{{J}_{1} ( Λ )}{{J}_{0} ( Λ )} ) \cdot {e}^{iwt} .$$

In very simplified case if a vessel of radius R, axis symmetric velocity profiles v(r,t) can be described in cylindrical coordinates by the empirical relationship:

v(r,t)≈vₘₐₓ*(1−(r/R)^{ξ})*ƒ(t)

where −1<(r/R)<1, ƒ(t) is a periodic function of heart beat frequency, which is driven by systolic pressure wave and it is time phase-shifted with respect to the cardiac cycle, and ξ represents the degree of blunting. For example, in 30 micron arterioles, there is a range of ξ 2.4-4 at normal flow rates. If ξ=2, a parabolic velocity distribution is obtained. Blunting would occur even in larger arterioles at low flow rates. The standard deviation d(v) can be calculated by:

$$rms ( dV ) = {v}_{max} * f ( t ) \sqrt{\frac{{\int }_{ }^{ } dv ( r ) * {r}^{2} * dr}{{\int }_{ }^{ } dv ( r ) * dr}} = \frac{ξ * {R}^{2}}{2 + ξ} * {v}_{max} * f ( t )$$

It can be shown that rms(dV) is proportional to the blood flow velocity. In terms of autocorrelation then decay time of autocorrelation function can be estimated by

$${τ}_{0} \approx \frac{1}{dV ( L )} .$$

Actually, the decay time of the process is very short and it means that high frequency component of the signal is closely is associated with the arterial blood flow signal.

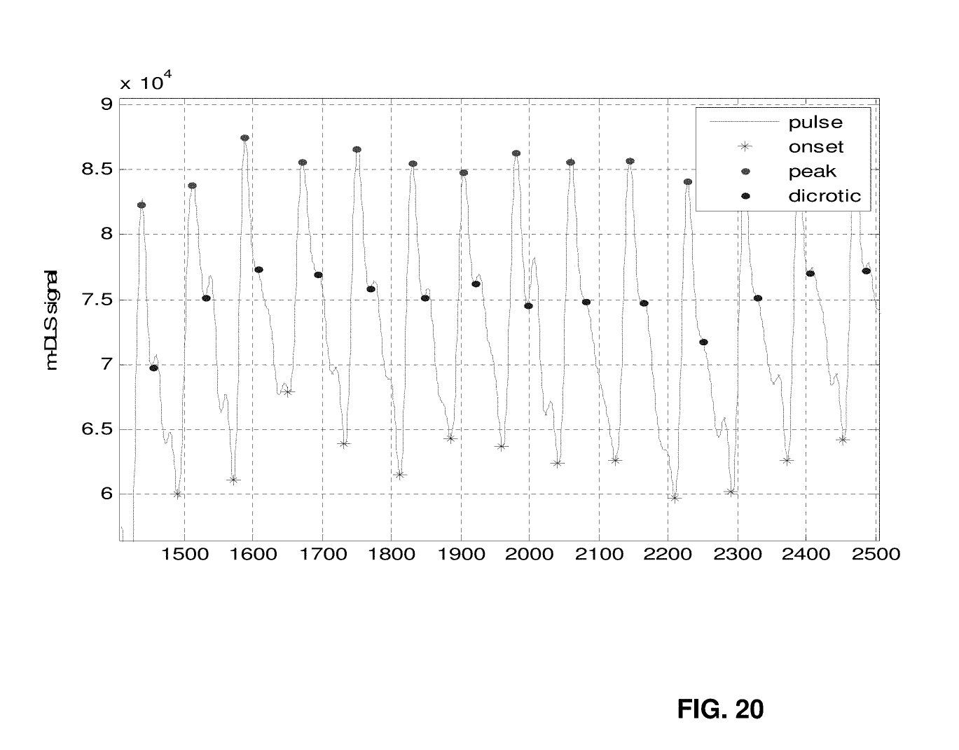

For small arterials (around 20 microns), the fluctuation of velocity from systolic to diastolic phases ranges from 1.5 mm/s to 2.5 mm/s. This results in a very significant fluctuation of standard deviation (Rms) during the systolic-diastolic cycle. Pulsatile signal, therefore, can be used for calculation of hemorheological parameters. Since the blood velocity changes follow the blood pressure wave, the mDLS signal reflects the fluctuation of the blood pressure. The morphologic characteristics of the pulse-wave allow for the measurement of blood pressure fluctuation by using the reflecting wave: Example of identification of onset, peak and dischrotic notch of the pulse-wave (FIG. 20)

### The Morphologic Structure of Measured Pulse Wave:

Since the mDLS sensor enables to measure those blood flow characteristics continuously and noninvasively the wave time behavioral pattern exhibits very prominent manifestations of hemodynamic changes related to the cardio-vascular dynamics. Next figure demonstrates this important characteristic of the mDLS based pulse shape. The figure display typical pulse shapes of three monitoring methods: invasive blood pressure (IBP), PPG, and mDLS. It can be clearly seen that the both mDLS and IBP based signals show all CV characteristics described above while the PPG based signal is much less sensitive and shows only two peaks (systolic and Dichotic notch). We utilize those important pulse features to characterize physiological normal and abnormal scenarios. There are other types of Hemodynamic Responses that are measured by using blood shear rate related speckle response. It includes the very low frequency components of the signal where the most of the information related to the RBC-endothelial cells interaction and middle frequency components related to capillary blood flow.

The physiological response (PR) on stimulus application is achieved by defining the following Response function:

PR=F(OHR(t),HR(t),OHR(HR(t)))

This RP can be juxtaposed to big data analysis or Individual follow-up to provide the Cardiovascular Fitness and Stress Indexes, ether continuously or by spot measurements. The using of PR function where one of OHR is measured together with o HR can provides a new indexes usable for characteristics of Physiological response for fitness and stress application.

1. A method for optically measuring a cardiovascular fitness and/or stress and/or physiological parameter specific to a mammalian subject, the method comprising:
a. illuminating a portion of the subject's skin to scatter partially or entirely coherent light off of moving red blood cells (RBCs) of the subject to induce a scattered-light time-dependent optical response;
b. receiving the scattered light by a photodetector(s) to generate an electrical signal descriptive of the induced scattered-light time-dependent optical response;
c. processing the scattered-light-optical-response-descriptive electrical signal or a product thereof to generate therefrom a time-dependent blood-shear-rate-descriptive signal wherein the processing is performed according to a function-transformation-algorithm that is dynamically adjusted over time in response to (i) a measured or predicted similarity between the time-dependent blood-shear-rate-descriptive signal and a blood-pressure-waveform; (ii) a measured or predicted presence or strength of blood-pressure-waveform-feature(s) within the time-dependent blood-shear-rate-descriptive signal; and
d. computing the cardiovascular fitness and/or stress and/or physiological parameter from the time-dependent blood-shear-rate-descriptive signal.

2. A method for optically measuring a cardiovascular fitness and/or stress and/or physiological parameter of a subject, the method comprising:
a. illuminating a portion of the subject's skin to scatter partially or entirely coherent light off of moving red blood cells (RBCs) of the subject to induce a scattered-light time-dependent optical response;
b. receiving the scattered light by a photodetector(s) to generate an electrical signal descriptive of the induced scattered-light time-dependent optical response;
c. for each transformation-function-algorithm of a plurality of transformation-function-algorithms, respectively processing the scattered-light-optical-response-descriptive electrical signal or a product thereof to generate therefrom a respective time-dependent blood-shear-rate-descriptive signal;
d. analyzing each time-dependent blood-shear-rate-descriptive signal to determine a presence or strength of pulsatile-waveform-feature(s) within the time-dependent blood-shear-rate-descriptive signal;
e. comparing the results of the analysis of each time-dependent blood-shear-rate-descriptive signal; and
f. computing, in accordance with the results of the comparing and from one or more of the time-dependent blood-shear-rate-descriptive signal or a mathematical function thereof, the cardiovascular fitness and/or stress and/or physiological parameter of the subject.

3. The method of claim 1 wherein the pulsatile-waveform-feature(s) any of the following parameters, or a relation therebetween: (i) a presence, temporal-location, shape or amplitude of a reflected-wave protrusion; (ii) a presence, temporal-location, shape or amplitude of a dicrotic notch; (iii) a presence, temporal-location, shape or amplitude of a vascular resistance wave protrusion; (iv) a systolic down-slope; (v) a diastolic down-slope; (v) a systolic upstroke slope; (vi) relative heights or time-delays between of any of an overall peak, reflected-wave marker, a dicrotic notch, and vascular resistance wave marker.

4. The method of claim 1 wherein the subject-specific parameter is selected from the group consisting of (i) a pulse; (ii) a heart-rate variability; (iii) a blood pressure; (iv) a stroke-volume; (iv) respiration rate; (iv) an apnea event; (v) a measure of the aortic valve functionality; (vi) the sympathetic system activity, systolic blood pressure, and vascular aging; (vii) a measure of the myocardium's ability to expel blood to the body height of the Dicrotic notch relative to the systolic peak, and (viii) the time delay between them; a measure of arterial stiffness and ability to resist blood flow; (ix) a measure of how fast myocardium relaxes at the end of systolic cycle; (x) a measure of how fast the myocardium relaxes at the end of diastole; (xi) a measure of stroke volume.

5. The method of claim 1 wherein:
i. the scattered light is received by first and second photodetectors respectively situated at first and second locations to respectively generate first and second scattered-light-optical-response-descriptive electrical signals; and
ii. the time-dependent blood-shear-rate-descriptive signal is derived from a difference between the first and second scattered-light-optical-response-descriptive electrical signals.

6. The method of claim 1 wherein the processing comprises subjecting the scattered-light-optical-response-descriptive electrical signal or a product thereof to at least one of an autocorrelation analysis and a power spectrum analysis.

7. A method for optically measuring a cardiovascular fitness and/or stress and/or physiological parameter of a subject, the method comprising:
a. illuminating a portion of the subject's skin to scatter partially or entirely coherent light off of moving red blood cells (RBCs) of the subject to induce a scattered-light time-dependent optical response;
b. receiving the scattered light by a photodetector(s) to generate an electrical signal descriptive of the induced scattered-light time-dependent optical response;
c. processing the scattered-light-optical-response-descriptive electrical signal or a product thereof to generate therefrom a time-dependent blood-shear-rate-descriptive signal; and
d. post-processing the time-dependent blood-shear-rate-descriptive signal by computing therefrom at least one or more of: (i) a systolic upstroke slope; (ii) a systolic downstroke slope; (iii) a diastolic downstroke slope; (iv) a stroke-volume;
(v) a time-interval between a pulse-peak and a peak of the reflected wave; (vi) a time-interval between a pulse-peak and a time of the dicrotic notch; (v) a time-interval between a pulse-peak and a time of the vascular-resistance wave; (v) a time-interval between a peak of the reflected wave and a time of the dicrotic notch; (vi) a time-interval between a marker of the reflected wave and a time of the vascular-resistance wave; and (vii) a time-interval between a time of the dicrotic notch and a time of the vascular-resistance wave.

8-15. (canceled)
